(19) United States
(12) Patent Application Publication
(10) Pub. No.: US 2026/0251633 A1
(43) Pub. Date: Aug. 27, 2026

(54) COVALENT TARGET ENGAGEMENT

(71) Applicant: Promega Corporation, Madison, WI (US)

(72) Inventors: Matthew ROBERS, Madison, WI (US); Cesear CORONA, Madison, WI (US); Jim VASTA, Madison, WI (US); Inhong HWANG, Madison, WI (US); Kelly TESKE, Madison, WI (US)

(21) Appl. No.: 19/421,964

(22) Filed: Dec. 16, 2025

Related U.S. Application Data
(60) Provisional application No. 63/734,593, filed on Dec. 16, 2024.

(51) Int. Cl. G01N 33/50; G01N 21/64; G01N 33/58

(52) U.S. Cl. CPC G01N 33/502; G01N 21/6428; G01N 33/582; G01N 2021/6439; G01N 2333/90241

(57) ABSTRACT

Provided herein are systems and methods for conducting covalent target engagement assays. In particular, cysteine-reactive tracers are provided for covalent binding to labeled target proteins, and signals produced by the interaction or proximity of the tracers and labeled target proteins are used to detect/quantify binding of a cysteine-reactive test agent.

### CROSS-REFERENCE

This application claims the benefit of U.S. Provisional Patent Application No. 63/734,593, Dec. 16, 2024; which is incorporated by reference herein in its entirety.

### SEQUENCE LISTING

The text of the computer readable sequence listing filed herewith, titled “PRMG_43979_202_SequenceListing.xml”, created Dec. 16, 2025, having a file size of 7,615 bytes, is hereby incorporated by reference in its entirety.

### FIELD

Provided herein are systems and methods for conducting covalent target engagement assays. In particular, cysteine-reactive tracers are provided for covalent binding to labeled target proteins, and signals produced by the interaction or proximity of the tracers and labeled target proteins are used to detect/quantify binding of a cysteine-reactive test agent.

### BACKGROUND

Covalent drugs offer enormous therapeutic potential, and the biopharmaceutical industry has increasing interest in covalent drug discovery. Currently, there is no broadly-applicable, microplate-based method to quantify covalent target engagement in live cells.

### SUMMARY

Provided herein are systems and methods for conducting covalent target engagement assays. In particular, cysteine-reactive tracers are provided for covalent binding to labeled target proteins, and signals produced by the interaction or proximity of the tracers and labeled target proteins are used to detect/quantify binding of a cysteine-reactive test agent.

In some embodiments, provided herein are system comprising: (a) a target protein tethered to a first component of a luminescent reporter; and (b) a cysteine-reactive tracer comprising second component of the luminescent reporter tethered to a cysteine-reactive group.

In some embodiments, provided herein are methods of quantifying the covalent binding of a cysteine-reactive test agent to a target protein, the method comprising: (a) contacting a target protein tethered to a first component of a luminescent reporter with the cysteine-reactive test agent; (b) contacting the target protein with a cysteine-reactive tracer comprising second component of the luminescent reporter tethered to a cysteine-reactive group, (c) detecting a luminescent signal from the luminescent reporter, wherein the amount of the luminescent signal emitted is inversely proportional to the amount of the cysteine-reactive test agent covalently bound to the target protein.

In some embodiments, the first component of the luminescent reporter is a donor lumiphore, and the second component of the luminescent reporter is an acceptor fluorophore, wherein light emitted at a first wavelength from the donor lumiphore is capable of exciting the acceptor fluorophore, resulting in emission of light from the acceptor fluorophore at a second wavelength. In some embodiments, the donor lumiphore is bioluminescent. In some embodiments, the bioluminescent donor lumiphore is a luciferase. In some embodiments, provided herein are the bioluminescent donor lumiphore comprises at least 70% (e.g., 70%, 75%, 80%, 85%, 90%, 95%, 100%, or ranges therebetween) sequence identity with SEQ ID NO: 1. In some embodiments, systems and methods further comprise a luminescent substrate for the bioluminescent donor lumiphore, wherein light is emitted at a first wavelength from the bioluminescent donor lumiphore in the presence of a luminescent substrate. In some embodiments, the luminescent substrate is an imidazopyrazine. In some embodiments, the imidazopyrazine is selected from coelenterazine, furimazine, and fluorofurimazine. In some embodiments, the donor lumiphore is fluorescent. In some embodiments, the fluorescent donor lumiphore is a small molecule fluorophore. In some embodiments, the fluorescent donor lumiphore is a fluorescent protein. In some embodiments, the acceptor fluorophore is a small molecule fluorophore.

In some embodiments, the first component of the luminescent reporter is a first component of a bioluminescent complex and the second component of the luminescent reporter is a second component of the bioluminescent complex, wherein upon formation of the bioluminescent complex by binding of the first component to the second component light is emitted from the bioluminescent complex in the presence of a luminescent substrate. In some embodiments, the first component of the bioluminescent complex comprises at least 70% (e.g., 70%, 75%, 80%, 85%, 90%, 95%, 100%, or ranges therebetween) sequence identity with SEQ ID NO: 2, and the second component of a bioluminescent complex comprises at least 70% (e.g., 70%, 75%, 80%, 85%, 90%, 95%, 100%, or ranges therebetween) sequence identity with SEQ ID NO: 3. In some embodiments, systems and methods further comprise a luminescent substrate for the bioluminescent complex. In some embodiments, the luminescent substrate is an imidazopyrazine. In some embodiments, the imidazopyrazine is selected from coelenterazine, furimazine, and fluorofurimazine.

In some embodiments, systems further comprise a cysteine-reactive test agent. In some embodiments, the cysteine-reactive test agent comprises a cysteine-reactive moiety (e.g., a functional group capable of forming a covalent bond to a thiol group of a cysteine) and a targeting moiety. In some embodiments, the cysteine-reactive group is selected from a maleimide moiety, an acrylamide moiety, a chloroacetamide moiety, a geminal dicyanocyclopropane carboxamide moiety, a bromo-dihydroisoxazole carboxamide moiety, and an azetidinyl oxadiazole moiety.

In some embodiments, the target protein and the cysteine-reactive tracer are within a cell. In some embodiments, methods comprise a step of expressing the target protein within the cell. In some embodiments, the target protein is present as a target fusion with the first component of the luminescent reporter, and the method further comprises a step of expressing the target fusion within the cell. In some embodiments, methods further comprise a step of adding the cysteine-reactive tracer extracellularly and allowing the cysteine-reactive tracer to enter the cell. In some embodiments, methods further comprise a step of adding the cysteine-reactive test agent extracellularly and allowing the cysteine-reactive test agent to enter the cell.

In some embodiments, provided herein are methods of quantifying the covalent binding of a cysteine-reactive test agent to a target protein within a cell, the method comprising: (a) providing a cell comprising a target protein tethered to a first component of a luminescent reporter; (b) contacting the cell with the cysteine-reactive test agent, and allowing the cysteine-reactive test agent to enter the cell and interact with the target protein; (b) lysing the cell to produce a cell lysate; (b) contacting the cell lysate with a cysteine-reactive tracer comprising second component of the luminescent reporter tethered to a cysteine-reactive group, and allowing the cysteine-reactive tracer to interact with the target protein; (c) detecting a luminescent signal from the luminescent reporter, wherein the amount of the luminescent signal emitted is inversely proportional to the amount of the cysteine-reactive test agent covalently bound to the target protein. In some embodiments, step (a) comprises expressing a fusion of the target protein and the first component of the luminescent reporter. In other embodiments, step (a) comprises contacting the cell with an agent capable of labeling the target protein with the first component of the luminescent reporter. In some embodiments, lysing the cell comprises mechanical lysis, chemical lysis, and/or enzymatic lysis.

### BRIEF DESCRIPTION OF THE DRAWINGS

FIG. 1. Cartoon depiction of a generic exemplary embodiment of a covalent target engagement assay.

FIG. 2. Graphs depicting BRET signal vs concentration of various cysteine-reactive test agents: (left to right) KRAS and AMG-510, BTK and ibrutinib, and SARS CoV2 Main Protease and nirmarevir.

FIG. 3. Graphs depicting BRET signal vs concentration and bar graphs comparing covalent compound knockdown fold-change for various cysteine-reactive test agents: Row 1 (left to right) domain only NLuc-USP7(207-650) and NLuc-USP7(207-650)(C315,510S) with XL177A; NLuc-ITK, NLuc-ITK(360-620), and NLuc-ITK(360-620)(C477S) with PF-0645469; and NLuc-FAK, NLuc-FAK(411-686), and NLuc-FAK(411-686)(C456,459,559,647S) with FAK-IN-2.

FIG. 4A-B. A) chemical structures, names, and excitation and emission wavelengths for various cysteine-reactive tracers investigated in BRET assay. B) A bar graph comparing the compound knockdown (for each cysteine-reactive tracer. The dashed line shows the desired threshold of 2-fold or greater.

FIG. 5A-B. A) Graph comparing potency of various cysteine-reactive tracers. B) Graph comparing the compound knockdown (assay window) at each concentration tested for the cysteine-reactive tracer investigated in A.

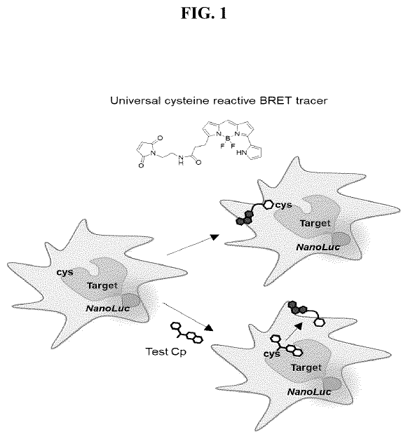

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “sample” is used in its broadest sense and includes a specimen or culture obtained from any source, an assay or reaction mixture, as well as biological and environmental samples. In particular, a sample is a liquid that comprises one or more proteins, including a target protein fusion. A sample may be a cell, comprise a cell, or be a cell lysate. A sample may be a biochemical mixture or solution comprising proteins. A sample may comprise a tissue or whole organism.

As used herein, the term “luminescence” refers to the emission of light from an agent or substance without incandescence (i.e., not as the result of heat). Examples of luminescence include fluorescence, bioluminescence, and chemiluminescence.

As used herein, the term “fluorescence” refers to the emission of electromagnetic radiation (e.g., light) by an agent or substance at an emission wavelength as a result of excitation of the agent or substance by irradiation at a different excitation wavelength. The terms “fluorescent moiety” and “fluorophore” are used interchangeably to refer to any molecular entity capable of emitting fluorescence in response to irradiation at an excitation wavelength (e.g., a fluorescent protein, a small molecule fluorophore, etc.).

As used herein, the term “bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light as it returns to its stable form.

As used herein, the term “lumiphore” broadly refers to any molecular entity capable of emitting light, whether through fluorescence, phosphorescence, or bioluminescence. In particular, it can apply to fluorophores, which emit light through fluorescence (light absorption and emission), and luciferases, which emit light through bioluminescence (chemical reaction).

As used herein, the term “luminescent substrate” refers to a chemical moiety or compound that can be placed in an excited electronic state (e.g., by a chemical or enzymatic reaction) and emits light as it returns to its electronic ground state.

As used herein, the term “imidazopyrazine” refers to a genus of luminescent substrates including “native coelenterazine” as well as synthetic (e.g., derivative or variant) and natural analogs thereof, such as furimazine, furimazine analogs (e.g., fluorofurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference in their entireties.

As used herein, the term “coelenterazine” refers to the naturally-occurring (“native”) imidazopyrazine of the structure:

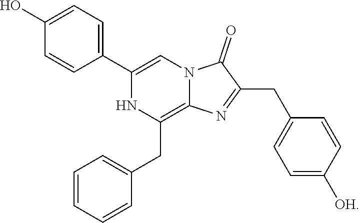 [SMILES: O=c1c(Cc2ccc(O)cc2)nc2c(Cc3ccccc3)[nH]c(-c3ccc(O)cc3)cn1-2]

As used herein, the term “furimazine” refers to the coelenterazine derivative of the structure:

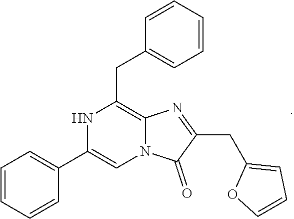 [SMILES: O=c1c(Cc2ccco2)nc2c(Cc3ccccc3)[nH]c(-c3ccccc3)cn1-2]

As used herein, the term “fluorofurimazine” refers to the furimazine derivative of the structure:

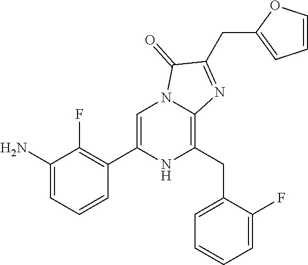 [SMILES: Nc1cccc(-c2cn3c(=O)c(Cc4ccco4)nc-3c(Cc3ccccc3F)[nH]2)c1F]

(U.S. application Ser. No. 16/548,214; incorporated by reference in its entirety).

As used herein, the term “resonance energy transfer” (“RET”) refers to the radiationless transmission of excitation energy from a first “donor” moiety to a second “acceptor” moiety. For example, in “Forster resonance energy transfer” (“FRET”), a donor fluorophore in an excited state transfers its energy to a proximal acceptor molecule by non-radiative dipole-dipole interaction. A description of FRET can be found in T. Forster, 1948, “Intermolecular Energy Migration and Fluorescence”, Ann. Phys., 2:55-75; and J. R. Lakowicz, 1999, “Principles of Fluorescence Spectroscopy”, 2nd ed. Plenum, New York. 367-394 (incorporated by reference in its entirety). Resonance energy transfer also includes luminescence resonance energy transfer, bioluminescence resonance energy transfer, chemiluminescence resonance energy transfer, and other similar types of energy transfer.

As used herein, the term “bioluminescence resonance energy transfer” (“BRET”) refers to the distance-dependent interaction in which energy is transferred from a donor bioluminescent protein/complex and substrate to an acceptor molecule without emission of a photon. The efficiency of BRET is dependent on the inverse sixth power of the intermolecular separation, making it useful over distances comparable with the dimensions of biological macromolecules (e.g., within 30-80 Å, depending on the degree of spectral overlap).

As used herein, the term “Oplophorus luciferase” (“OgLuc”) refers to a luminescent polypeptide having significant sequence identity, structural conservation, and/or the functional activity of the luciferase produced by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 1 (NANOLUC), which comprises 10 β strands (β1, β2, β3, β4, β5, β6, β7, β8, β9, β10) and utilize substrates such as coelenterazine or a coelenterazine derivative or analog to produce luminescence.

As used herein, the term “reporter” refers to a molecular entity or group (e.g., pair) of molecular entities that produce(s) a detectable signal that correlates with a molecular event.

The term “tracer” as used herein refers to a molecular entity comprising a target engagement moiety (e.g., cysteine-reactive moiety) and a detectable label (e.g., fluorophore that can be detected/monitored in an assay (e.g., a covalent target engagement assay). The term “covalent tracer” refers to a detectably labelled molecular entity that binds covalently to a target.

As used herein, the term “system” refers to a collection of compositions grouped together in any suitable manner (e.g., physically associated, within the same fluid (e.g., reaction mixture, cell lysate, etc.), body (e.g., cell), packaged together (e.g., in a kit), etc.) for a particular purpose.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

As used herein, the terms “tethering” or “tethered” refer to stably connecting or linking two entities by any suitable covalent or non-covalent linkage. A first protein, polypeptide, or peptide entity can be tethered to a second protein, polypeptide, or peptide entity by genetic fusion (e.g., directly or via a peptide linker). The entities can be tethered by the non-covalent interaction of affinity agents/entities attached. The entities can be tethered by a covalent linkage formed between reactive agents/moieties on each entity (e.g., click chemistry, HALOTAG, etc.). Unless specified otherwise, embodiments herein are not limited by the type of tether or the types of entities (e.g., small molecule, peptide, protein, antibody, lipid, etc.) that are tethered together.

As used herein, the term “linker” refers to a chemical moiety that serves as a covalent tether between the two entities. A linker may be a peptide (e.g., in the case of a fusion of two peptide/polypeptide entities), a carbon chain (e.g., optionally interrupted and/or substituted by one or more nitrogen atoms, oxygen atoms, carbonyl groups, rings, or other functional groups), a polymer (e.g., PEG), or any other chemical moiety capable of tethering two entities. A linker may tether a chemical moiety (e.g., reactive group, fluorophore, affinity label, etc.) to a component of an assay system (e.g., antibody, protein target, analyte, etc.). Unless specified otherwise, embodiments herein are not limited by the type of tether or the types of entities (e.g., small molecule, peptide, protein, antibody, lipid, etc.) that are tethered together.

As used herein the term “BODIPY dye,” or equivalents thereof, refers to a fluorescent compound having a boron-dipyrromethene core:

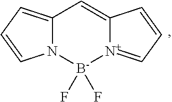 [SMILES: F[B-]1(F)n2cccc2C=C2C=CC=[N+]21]

which can be optionally substituted, for example with a linker to a cysteine reactive moiety, e.g.:

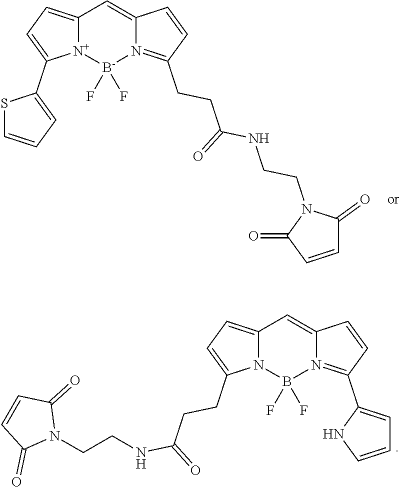

### DETAILED DESCRIPTION

Provided herein are systems and methods for conducting covalent target engagement assays. In particular, cysteine-reactive tracers are provided for covalent binding to labeled target proteins, and signals produced by the interaction or proximity of the tracers and labeled target proteins are used to detect/quantify binding of a cysteine-reactive test agent.

Covalent drugs are a class of pharmaceuticals that exert their therapeutic effects by forming covalent bonds with specific biomolecule targets, typically target proteins. Unlike traditional drugs, which bind reversibly to their targets, covalent drugs form a stable, irreversible bond with the target, often modifying its function. This mechanism of action can offer several advantages, such as enhanced potency, longer duration of action, and potentially overcoming resistance mechanisms. Covalent drugs form a permanent bond with their target molecules, often through a chemical reaction between the drug and a specific amino acid residue (e.g., cysteine) in the target protein. The ability of a covalent drug to selectively target a specific protein or enzyme is essential for minimizing off-target effects. The development of covalent drugs requires careful design to ensure selectivity, as the irreversible binding can lead to unwanted off-target effects if the drug binds to unintended proteins. Furthermore, there is a risk of toxicity if the covalent bond forms in a non-specific manner. Additionally, it is challenging to assess whether the covalent drug has formed a covalent bond with the target or merely made a stable non-covalent interaction.

Cysteine-reactive covalent drugs specifically target the thiol (—SH) group of cysteine residues within proteins. Cysteine residues are nucleophilic, meaning they have a high affinity for electrophilic groups, making them ideal sites for covalent modification. The modification of cysteine residues is a well-established mechanism for covalent drug binding, and this class of drugs has shown promise in treating various diseases, especially those driven by dysregulated proteins. Cysteine-reactive covalent drugs typically contain (a) a targeting moiety that is specific for a binding site on the target protein, and (b) electrophilic warhead moiety (a cysteine reactive chemical group) that can form a covalent bond with the thiol group on a cysteine at or near the binding site. Upon interaction of the drug with the binding site, the electrophilic warhead moiety reacts with the cysteine thiol (—SH), forming a stable covalent bond. This can lead to the inhibition of the target protein's activity or induce conformational changes that affect its function. Design and testing of cysteine-reactive covalent drugs is a current challenge in the field. Embodiments herein address that challenge.

Embodiments herein utilize generic cysteine-reactive probes (e.g., tracers comprising (1) a cysteine-reactive moiety and (2) a first component of a luminescent reporter (e.g., a RET system or structural complementation system)) to measure covalent target engagement for any target protein. A target protein tethered to a first component of the luminescent reporter is contacted with a covalent, cysteine-reactive test agent (e.g., added to the live cell medium), and a non-selective cysteine reactive tracer (e.g., cysteine-reactive moiety tethered to a second component of the luminescent reporter) is added after test agent addition; luminescence of the luminescent reporter is triggered (e.g., a substrate is added, the system is exposed to light at an appropriate excitation wavelength, etc.), and luminescence (e.g., BRET, FRET, direct luminescence from an assembled luminescent complex, etc.) is detected. The non-selective cysteine reactive tracer will react with cysteine residues on any proteins, but only binding to the tagged target protein will be detected by the two-component luminescent reporter. Binding of the test agent results in competition with the non-specific tracer. Increased covalent binding of the cysteine-reactive test agent to the target protein will result in decreased binding of the non-selective cysteine reactive tracer to the target protein and therefore decreased signal from the two-component luminescent reporter.

Some embodiments herein utilize generic cysteine-reactive RET probes (e.g., tracers comprising (1) a cysteine-reactive moiety and (2) an acceptor fluorophore) to measure covalent target engagement for any target protein tethered (e.g., fused, chemically conjugated, or otherwise linked) to a donor lumiphore, e.g., a bioluminescent protein, a fluorophore, etc. A luminescently-tagged target protein (e.g., expressed in cells) is contacted with a covalent, cysteine-reactive test agent (e.g., added to the live cell medium), and a non-selective cysteine reactive RET tracer (e.g., cysteine-reactive moiety tethered to an acceptor fluorophore) is added after test agent addition; luminescence of the donor lumiphore is triggered (e.g., a substrate for a bioluminescent donor is added, the donor lumiphore is exposed to light of its excitation wavelength, etc.), and RET from the donor lumiphore to the acceptor fluorophore is detected. The non-selective cysteine reactive RET tracer will react with cysteine residues on any proteins, but only binding to the luminescently-tagged target protein will be detected by RET. Binding of the test agent results in competition with the non-specific RET tracer. Increased covalent binding of the cysteine-reactive test agent to the target protein will result in decreased binding of the non-selective cysteine reactive RET tracer to the target protein and therefore decreased RET signal.

Certain embodiments herein utilize donor-fluorophore-labeled target proteins and generic cysteine-reactive FRET probes (e.g., tracers comprising (1) a cysteine-reactive moiety and (2) a fluorophore) to measure covalent target engagement. A fluorescently-tagged target protein (e.g., expressed in cells) is contacted with a covalent, cysteine-reactive test agent (e.g., added to the live cell medium), and a non-selective cysteine reactive FRET tracer (e.g., cysteine-reactive moiety tethered to an acceptor fluorophore) is added after test agent addition, and FRET from the donor fluorophore to the acceptor fluorophore is detected. The non-selective cysteine reactive FRET tracer will react with cysteine residues on any proteins, but only binding to the fluorescently-tagged target protein will be detected by FRET. Binding of the test agent results in competition with the non-specific FRET tracer. Increased covalent binding of the cysteine-reactive test agent to the target protein will result in decreased binding of the non-selective cysteine reactive FRET tracer to the target protein and therefore decreased FRET signal.

Other embodiments herein utilize bioluminescently-labeled target proteins and generic cysteine-reactive BRET probes (e.g., tracers comprising (1) a cysteine-reactive moiety and (2) an acceptor fluorophore) to measure covalent target engagement. A bioluminescently-labeled target protein (e.g., expressed (in cells) as a fusion or otherwise linked together) is contacted with a covalent, cysteine-reactive test agent (e.g., added to the live cell medium), and a non-selective cysteine reactive BRET tracer (e.g., cysteine-reactive moiety tethered to an acceptor fluorophore) is added after test agent addition, and BRET from the donor bioluminescent tag to the acceptor fluorophore is detected. The non-selective cysteine reactive BRET tracer will react with cysteine residues on any proteins, but only binding to the bioluminescently-tagged target protein will be detected by BRET. Binding of the test agent results in competition with the non-specific BRET tracer. Increased covalent binding of the cysteine-reactive test agent to the target protein will result in decreased binding of the non-selective cysteine reactive BRET tracer to the target protein and therefore decreased BRET signal.

Additional embodiments herein utilize generic cysteine-reactive probes (e.g., tracers comprising (1) a cysteine-reactive moiety and (2) a first component of a bioluminescent complex (e.g., a peptide or polypeptide component) to measure covalent target engagement for any target protein tethered to a second component of the bioluminescent complex (e.g., a polypeptide or peptide component). A target protein tethered to a first component of the bioluminescent complex is contacted with a covalent, cysteine-reactive test agent (e.g., added to the live cell medium), and a non-selective cysteine reactive tracer (e.g., cysteine-reactive moiety tethered to the second component of the bioluminescent complex) is added after test agent addition; bioluminescence of the bioluminescent complex is triggered (e.g., a substrate is added,), and bioluminescence from an assembled bioluminescent complex is detected. The non-selective cysteine reactive tracer will react with cysteine residues on any proteins, but only binding to the tagged target protein will be detected by formation of the two-component bioluminescent complex. Binding of the test agent results in competition with the non-specific tracer. Increased covalent binding of the cysteine-reactive test agent to the target protein will result in decreased binding of the non-selective cysteine reactive tracer to the target protein, decreased interaction between the first and second components of the bioluminescent complex, decreased bioluminescent complex formation, and therefore decreased signal from the two-component bioluminescent complex.

Methods and systems herein comprise (1) cysteine-reactive test agent, (2) a target protein fused to the first component of a luminescent reporter (e.g., a bioluminescent protein, a donor fluorophore, a first component of a bioluminescent complex, etc.), and (3) a cysteine-reactive tracer (e.g., a cysteine-reactive moiety linked to a second component of the luminescent reporter (e.g., an acceptor fluorophore, a second component of a bioluminescent complex). Each of these components is described in more detail below. Any combinations of the various components provided herein may find use in embodiments of the present technology.

Embodiments herein find use in testing cysteine-reactive test agents for binding to target proteins.

### I. Test Agent

In some embodiments, a test agent is specific for a cysteine-containing target site on a target protein. In some embodiments, the test agent is cysteine reactive, such that it forms a covalent bond with the thiol of the target site upon binding thereto. In some embodiments, a test agent comprises a cysteine-reactive moiety (e.g., a functional group capable of forming a covalent bond to a thiol group of a cysteine) and a targeting moiety (e.g., a molecular moiety capable of binding to (e.g., non-covalently) the target site on a protein. In other embodiments, a test agent is capable of covalently binding to a target-site cysteine on a target protein but does not comprise a separate targeting and cysteine-reactive moieties.

A test agent (or the targeting moiety thereof) may be any suitable molecule capable of binding to a target site on a target protein, such as a small molecule, peptide, antibody, antibody fragment, etc.

Exemplary test agents (or targeting moieties thereof) include but are not limited to Hsp90 inhibitors, kinase inhibitors, MDM2 inhibitors, compounds targeting proteins containing human BET bromodomains, compounds targeting cytoplasmic signaling protein FKBP12, HDAC inhibitors, human lysine methyltransferase inhibitors, angiogenesis inhibitors, immunosuppressive compounds, compounds targeting aryl hydrocarbon receptor (AHR), etc.

In certain embodiments, a test agent (or the targeting moiety thereof) is designed to and/or is capable of binding to one or more kinases, BET bromodomain-containing proteins, cytoplasmic signaling proteins (such as FKBP12), nucleoproteins, histone deacetylases, lysine methyl transferase, protein regulating angiogenesis, proteins regulating immune response, aromatic hydrocarbon receptors (AHRs), estrogen receptors, androgen receptors, glucocorticoid receptors, or transcription factor (e.g., SMARCA4, SMARCA2, TRIM24).

In certain embodiments, a test agent (or the targeting moiety thereof) is designed to and/or is capable of binding to one or more kinases, such kinases may include, but not limited to Tyrosine kinases (for example, AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFRE3, FGFR4, FGR, FLT1, FLT3, FLT4, FRK, FYN, GSG2, HCK, HRAS, HSP90, IGF1R, ILK, INSR, INSRR, IRAK4, ITK, JAK1, JAK2, JAK3, KDR, KIT, KRAS, KSP, KSR1, LCK, LMTK2, LMTK3, LTK, LYN, MATK, MERTK, MET, MLTK, MST1R, MUSK, NPR1, NRAS, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1 or ZAP70), Serine/threonine kinase (such as Casein Kinase 2, protein kinase A, protein kinase B, protein kinase C. Raf kinase, CaM kinase, AKT1, AKT2, AKT3, ALK1, ALK2, ALK3, ALK4, Aurora A. Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MEK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-β, TLK2, TSSK1, TSSK2, MLK1 or MLK2), cyclin-dependent kinases (such as Cdkl-Cdk11) and Leucine-rich repetitive kinase (such as LRRK2).

Other target proteins that find use in the embodiments herein include EGFR (Epidermal Growth Factor Receptor), BCR-ABL, PTEN (Phosphatase and tensin homolog), MEK1/2, PI3K, AKT, Matrix Metalloproteinases (MMPs), HIV protease, HCV NS3 Protease, Influenza Neuraminidase, Caspases, Renin, Adenylyl cyclase, dehydrogenases (e.g., lactate dehydrogenase (LDH), etc.), Cyclooxygenases (e.g., COX-1, COX-2, etc.), G-Protein Coupled Receptors (GPCRs) (e.g., Beta-adrenergic receptors, Dopamine receptors, Serotonin receptors, etc.), ion channels (e.g., voltage-gated sodium channels, potassium channels (e.g., Kv channels), Calcium channels (e.g., L-type calcium channels), etc.), Nuclear Receptors (e.g., Estrogen receptor, Glucocorticoid receptor, Peroxisome proliferator-activated receptor (PPAR), etc.), Tyrosine Kinase Receptors (e.g., Insulin receptor, VEGF (vascular endothelial growth factor) receptor, etc.), Sodium/Potassium ATPase, Serotonin Transporter (SERT), Dopamine Transporter (DAT), TNF-α (Tumor Necrosis Factor-alpha), Interleukins (e.g., IL-1, IL-6, IL-12), Immune Checkpoint Proteins (e.g., PD-1, CTLA-4, etc.), C5, Bcl-2, IAPs (Inhibitor of Apoptosis Proteins), P-glycoprotein, SGLT2, HDAC1/2/3/6, farnesyltransferase, sirtuins, HSP90, prolyl hydroxylases, etc. In some embodiments, one or more of the above proteins, a related protein, and/or a different target protein (a therapeutic drug target) is provided (e.g., expressed in a cell) as a fusion protein with a bioluminescent protein (e.g., a luciferase (e.g., NANOLUC, etc.), etc.) In some embodiments, a test agent comprises a cysteine-reactive moiety and a targeting moiety capable of and/or designed/intended to interact with (e.g., bind) one or more of the above proteins, a related protein, and/or a different target protein (a therapeutic drug target).

In some embodiments, in addition to a targeting moiety (e.g., a molecular moiety capable of interacting with a target protein (e.g., binding to (typically non-covalently)), a cysteine-reactive test agent for use in embodiments herein comprises a cysteine-reactive moiety. In some embodiments, a cysteine-reactive moiety of a test agent is an electrophilic group. In some embodiments, the cysteine-reactive moiety is selected from a maleimide moiety, an acrylamide moiety, a chloroacetamide moiety, a geminal dicyanocyclopropane carboxamide moiety, a bromo-dihydroisoxazole carboxamide moiety, and an azetidinyl oxadiazole moiety. In some embodiments, a cysteine-reactive test agent comprises a structure of:

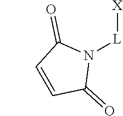

(test agent with maleimide cysteine-reactive moiety);

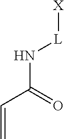

(test agent with acrylamide cysteine-reactive moiety);

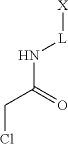

(test agent with chloroacetamide cysteine-reactive moiety);

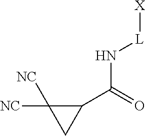

(test agent with geminal dicyanocyclopropane carboxamide cysteine-reactive moiety);

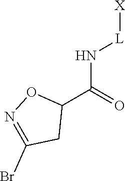

(test agent with bromo-dihydroisoxazole carboxamide cysteine-reactive moiety); and

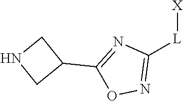

(test agent with azetidinyl oxadiazole cysteine-reactive moiety);
wherein L is a linker, and X is a targeting moiety.

In some embodiments, a cysteine-reactive moiety and targeting moiety are directly covalently bonded to form a test agent (e.g., test agent comprises a cysteine-reactive moiety connected to a targeting moiety by a single covalent bond). In other embodiments, the cysteine-reactive moiety and targeting moiety are tethered to each other by a linker (L). Exemplary chemical linkers may comprise functional groups selected alkylene, arylene, —O—, —NH—, carbamate, and —C(O)— groups. For example, linkers may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ) linkages, and the like.

In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)).

In some embodiments, linker L of a test agent comprises —(CH₂)ₓNHC(O)(CH₂)_{y}—, wherein x and y are independently selected from 0-6.

In some embodiments, linker L of a test agent comprises a linker selected from:

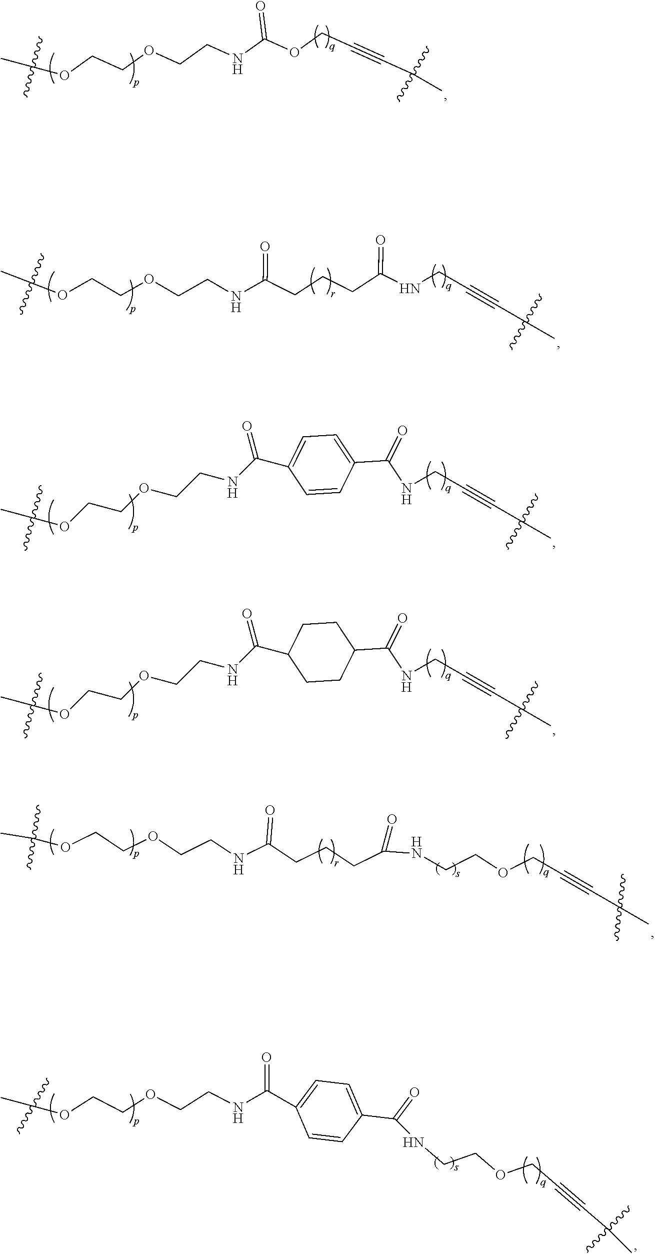

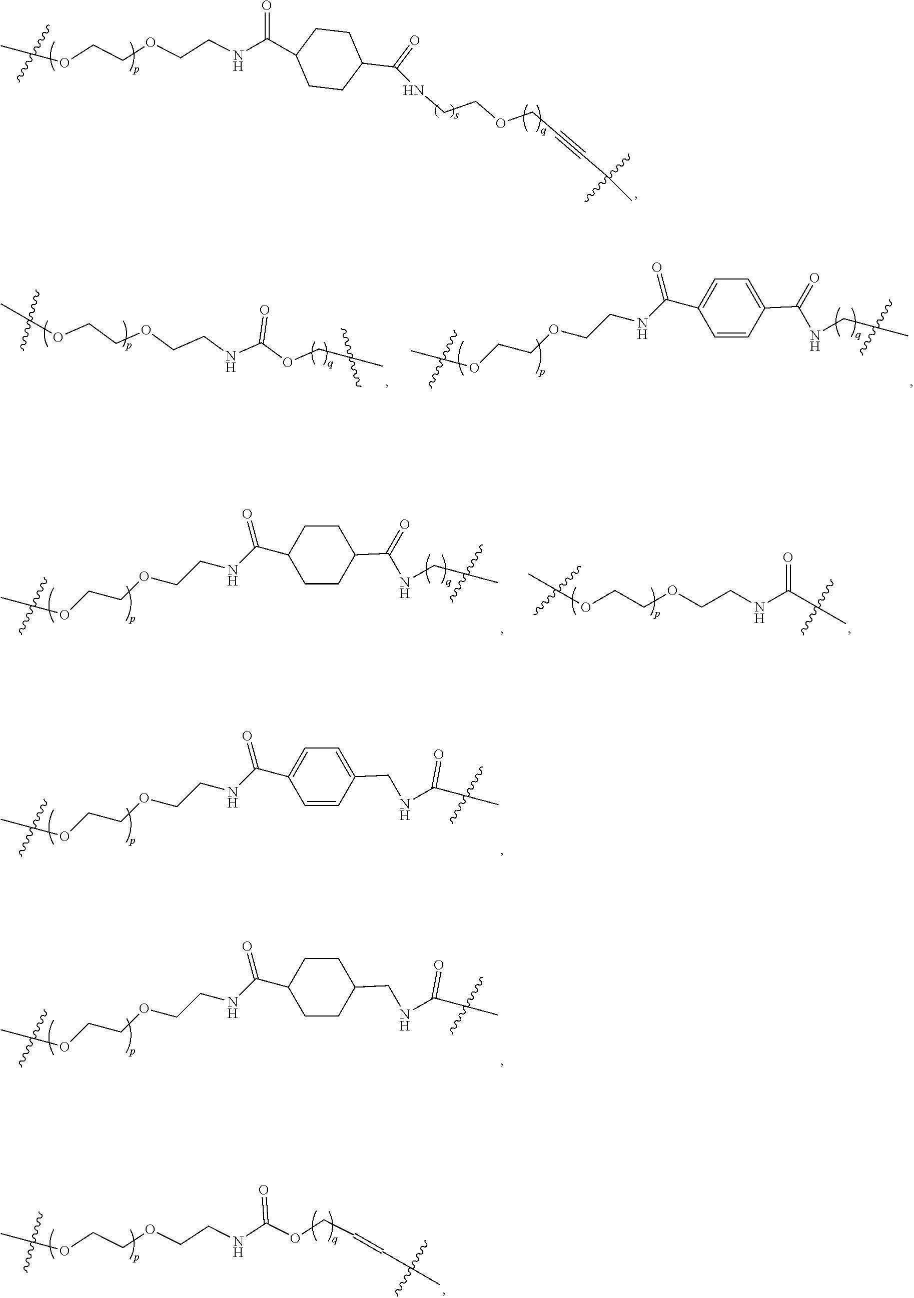

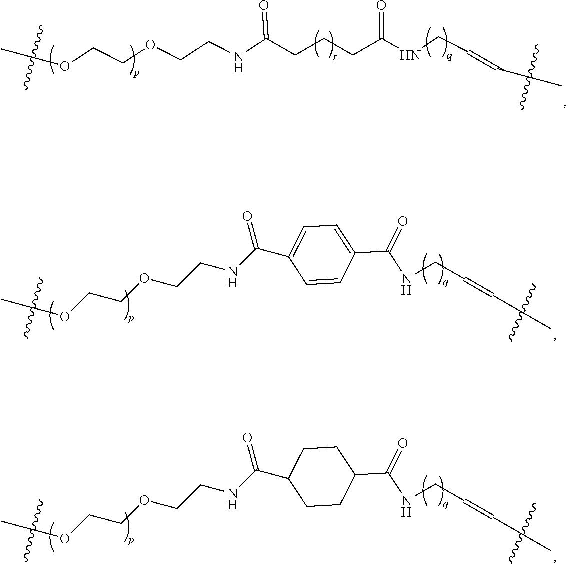

wherein p, q, r, and s are each independently 1-6. Other linkers suitable for tethering a cysteine-reactive moiety to a targeting moiety are understood in the field.

### II. Target Protein

In some embodiments provided herein are target proteins (e.g., potentially druggable targets) tethered to a first component of a luminescent reporter (e.g., donor lumiphore (e.g., bioluminescent protein or donor fluorophore) or first component of a bioluminescent complex). Suitable target proteins may be those listed above, any therapeutically useful target protein, or any other protein of interest. In some embodiments, the target protein is tethered (e.g., expressed as a fusion, chemically conjugated, non-covalently linked by affinity molecules, etc.) to the first component of the luminescent reporter. In some embodiments, the target protein is provided (e.g., expressed in a cell) as a fusion with the first component of a luminescent reporter.

### A. Donor Lumiphore

In some embodiments, the first component of the luminescent reporter is a lumiphore (e.g., bioluminescent protein or donor fluorophore).

### 1. Bioluminescent Protein

In some embodiments, the lumiphore is a bioluminescent protein (e.g., fused or otherwise linked to the target protein). In some embodiments, a bioluminescent protein is a luciferase. Suitable luciferases include those selected from the group consisting of: Photinus pyralis or North American firefly luciferase; Luciola cruciata or Japanese firefly or Genji-botaru luciferase; Luciola italic or Italian firefly luciferase; Luciola lateralis or Japanese firefly or Heike luciferase; N. nambi luciferase; Luciola mingrelica or East European firefly luciferase; Photuris pennsylvanica or Pennsylvania firefly luciferase; Pyrophorus plagiophthalamus or Click beetle luciferase; Phrixothrix hirtus or Railroad worm luciferase; Renilla reniformis or wild-type Renilla luciferase; Renilla reniformis Rluc8 mutant Renilla luciferase; Renilla reniformis Green Renilla luciferase; Gaussia princeps wild-type Gaussia luciferase; Gaussia princeps Gaussia-Dura luciferase; Cypridina noctiluca or Cypridina luciferase; Cypridina hilgendorfii or Cypridina or Vargula luciferase; Metridia longa or Metridia luciferase; TurboLuc (Auld et al. Biochemistry 2018, 57, 31, 4700-4706: incorporated by reference in its entirety); Nano-lanterns (Suzuki et al. Nature Communications volume 7, Article number: 13718 (2016); incorporated by reference in its entirety); and Oplophorus luciferase (e.g., Oplophorus gracilirostris (OgLuc luciferase), Oplophorus grimaldii, Oplophorus spinicauda, Oplophorus foliaceus, Oplophorus noraezeelandiae, Oplophorus typus, Oplophorus noraezelandiae or Oplophorus spinous). In some embodiments, a bioluminescent protein (in a fusion with a target protein) is a luciferase derived from the OgLuc luciferase, such as the NANOLUC® luciferase (Promega Corporation; U.S. Pat. Nos. 8,557,970; 8,669,103; incorporated by reference in their entireties) and engineered luciferases related thereto. PCT Appln. No. PCT/US2010/033449, U.S. Pat. No. 8,557,970, PCT Appln. No. PCT/2011/059018, and U.S. Pat. No. 8,669,103 (each of which is incorporated by reference in their entirety and for all purposes) describe compositions and methods comprising bioluminescent polypeptides; such polypeptides find use in embodiments herein (e.g., as a fusion with a target protein). In some embodiments, target protein fusions comprise a luciferase of SEQ ID NO: 1, or having at least 60% (e.g., 60%, 65%, 70%, 75%, 80%, 85%, 90%, 95%, 96%, 97%, 98%, 99%, 100%, or ranges therebetween) sequence identity with SEQ ID NO: 1. In some embodiments, target protein fusions comprise a luciferase that is a circularly permuted version of a natural or modified bioluminescent protein (See, e.g., U.S. Pat. No. 10,774,364; incorporated by reference in its entirety).

Suitable substrates are provided that emit light upon interaction with the bioluminescent proteins described herein. Suitable substrates for the target bioluminescent protein fusions herein will be understood. For example, firefly luciferin, with the structure:

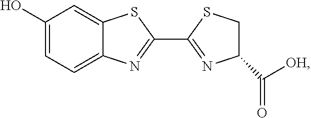 [SMILES: O=C(O)[C@H]1CSC(c2nc3ccc(O)cc3s2)=N1]

is the luciferin found in many Lampyridae species, and is the substrate of beetle luciferases.

Latia luciferin, with the structure:

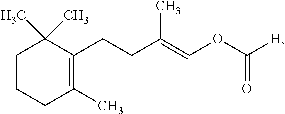

is from the freshwater snail Latia neritoides.

Bacterial luciferin, with the structure:

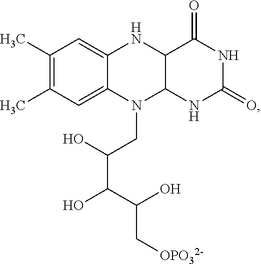

finds use as a substrate for many bacterial luciferases.

Coelenterazine of the structure:

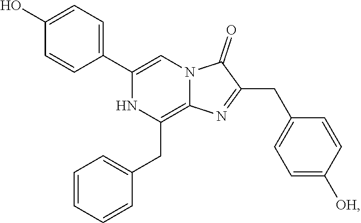 [SMILES: O=c1c(Cc2ccc(O)cc2)nc2c(Cc3ccccc3)[nH]c(-c3ccc(O)cc3)cn1-2]

is found in radiolarians, ctenophores, cnidarians, squid, brittle stars, copepods, chaetognaths, fish, and shrimp, and is the substrate for the luciferases of those organisms. Variants and derivatives of coelenterazine, such as furimazine and fluorofurimazine find use in embodiments herein (e.g., with Oplophorus-derived bioluminescent proteins and complexes).

Other substrates include those of dinoflagellates:

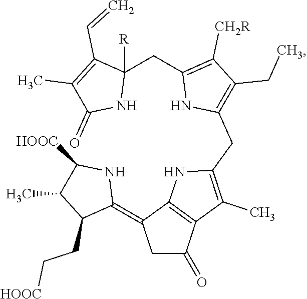

Vargulin (cypridin luciferin):

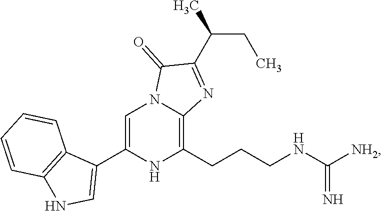 [SMILES: CC[C@H](C)c1nc2c(CCCNC(=N)N)[nH]c(-c3c[nH]c4ccccc34)cn-2c1=O]

and
N. nambi:

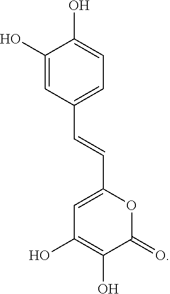 [SMILES: O=c1oc(/C=C/c2ccc(O)c(O)c2)cc(O)c1O]

Pairing of appropriate bioluminescent proteins with lumiphores is understood in the field. In particular embodiments, a bioluminescent protein is provided in a fusion with a target protein, and the bioluminescent protein utilizes an imidazopyrazine lumiphore, such as coelenterazine, furimazine, hydrofurimazine, or fluorofurimazine (U.S. application Ser. No. 16/548,214; incorporated by reference in its entirety). In some embodiments, provided herein is (1) a fusion of a target protein and an Oplophorus-derived polypeptide (e.g., NANOLUC) and (2) an imidazopyrazine lumiphore (e.g., coelenterazine, furimazine, hydrofurimazine, fluorofurimazine, etc.). In some embodiments, systems and methods herein comprise an imidazopyrazine lumiphore such as native coelenterazine, furimazine, hydrofurimazine, fluorofurimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); and U.S. Pat. No. 8,669,103; the disclosures of which are incorporated by reference herein in their entireties.

In some embodiments, the substrate emits light upon interaction with the bioluminescent protein. In some embodiments, the substrate emits light in the visible light spectrum (e.g., about 400 to about 700 nm (e.g., 400 nm, 425 nm, 450 nm, 475 nm, 500 nm, 525 nm, 550 nm, 575 nm, 600 nm, 625 nm, 650 nm, 675 nm, 700 nm, or ranges therebetween). In some embodiments, the substrate emits light of a wavelength between 400 and 500 nm (e.g., 400 nm, 410 nm, 420 nm, 430 nm, 440 nm, 450 nm, 460 nm, 470 nm, 480 nm, 490 nm, 500 nm, or ranges therebetween).

In some embodiments, a bioluminescent protein (e.g., in a fusion herein) is selected from NANOLUC (e.g., thermostable (TS) and non-thermostable variants), TURBOLUC, Firefly luciferase (FLuc), Ultra-Glo recombinant luciferase, Renilla luciferase (RLuc), Gaussia luciferase (GLuc), Click Beetle luciferase, and Fungal luciferase (Luz). Accordingly, each luciferase-based biosensor is used with its key cognate substrates including, but not limited to: Furimazine (Fz), Fluoro-Fz (FFz), Cephalo-Fz (CFz/CFz-9), coelenterazine (Cz), D-Luciferin, etc.

### 2. Fluorophore Donor

In some embodiments, the lumiphore is a donor fluorophore. A donor lumiphore is any entity capable of being tethered to the target protein (e.g., fused, chemically-conjugated, non-covalently linked by affinity molecules, etc.) and emitting at a suitable emission wavelength (e.g., upon excitation) to excite an acceptor fluorophore. Suitable donor fluorophores include fluorescent proteins and small molecule fluorophores (e.g., dyes). In some embodiments, the donor fluorophore is green fluorescent protein, organic dye, or lanthanide chelate.

In some embodiments, the donor fluorophore is a fluorescent protein is selected from yellow fluorescent protein (YFP), green fluorescent protein (GFP), cyan fluorescent protein (CFP), red fluorescent protein (RFP), umbelliferone, fluorescein, fluorescein isothiocyanate, rhodamine, dichlorotriazinylamine fluorescein, cyanines, dansyl chloride, phycocyanin, and phycoerythrin.

In some embodiments, the donor fluorophore is a small molecule (e.g., molecular weight less than 3,000 daltons, <2,500 daltons, <2,000 daltons, <1,500 daltons, <1,000 daltons, <900 daltons, <800 daltons, <700 daltons, <600 daltons). In such embodiments, the fluorophore is tethered to the target protein by a suitable conjugation technique, as discussed herein. Suitable small-molecule donor fluorophores for use in embodiments herein include, but are not limited to: stilbazolium derivatives (Marquesa et al. Mechanism-Based Strategy for Optimizing HALOTAG Protein Labeling. ChemRxiv. Cambridge: Cambridge Open Engage; 2021; incorporated by reference in its entirety), xanthene derivatives (e.g., fluoresceins, rhodamines, rhodols, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), CF dye (Biotium), BODIPY (Invitrogen), ALEXA FLUOR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), etc.

### B. Component of a Bioluminescent Complex

In some embodiments, the first component of the luminescent reporter is a first component of a bioluminescent complex. A bioluminescent complex comprises two or more components (e.g., peptides and/or polypeptides) that combine through structural complementation to form a complex that is capable of activating emitting light upon interaction with a luminescent substrate. The complex emits significantly more light in the presence of the substrate than any incomplete set of the components. In some embodiments, a bioluminescent complex is formed from fragments (e.g., peptide(s) and/or polypeptide(s)) of a luciferase enzyme. In some embodiments, a bioluminescent complex is a circularly permuted version of a natural or modified bioluminescent component (e.g., formed from two fragments of a circularly permuted luciferase); See, e.g., U.S. Pat. No. 10,774,364; incorporated by reference in its entirety. PCT Appln. Nos. PCT/US14/26354, PCT/US19/036844, and PCT/US20/62499; U.S. Pat. No. 9,797,889; U.S. patent application Ser. No. 16/439,565; and U.S. Pub. No. 2021/0262941 (each of which is herein incorporated by reference in their entirety and for all purposes) describe compositions (e.g., first and second components) and methods for the assembly of bioluminescent complexes; such complexes, and the peptide and polypeptide components thereof, find use in embodiments herein and can be used in conjunction with the systems and methods described herein.

In some embodiments, first and second components (and optionally third components) of the luminescent reporters in the systems and methods herein are bipartite or multipartite bioluminescent complexes, such as those derived from the commercially available NANOLUC protein (Promega), and/or the NANOBIT (Promega) or NANOTRIP structural complementation systems. The native Oplophorus gracilirostris luciferase (OgLuc) and commercially-available NANOLUC luciferase (Promega Corporation) each comprise polypeptides of 10 β (beta) strands (β1, β2, β3, β4, β5, β6, β7, β8, β9, β10). U.S. Pat. No. 9,797,889 (herein incorporated by reference in its entirety) describes development and use of a complementation system comprising a β1-9-like polypeptide and a β10-like peptide (certain OgLuc/NANOLUC-based polypeptide and peptide sequences in polypeptide and peptide sequences in U.S. Pat. No. 9,797,889 differ from the corresponding sequences in NANOLUC and wild-type native OgLuc). Similarly, U.S. application Ser. No. 16/439,565 (herein incorporated by reference in its entirety) describes the development and use of a complementation systems comprising two or more OgLuc/NANOLUC peptides and/or polypeptides (certain OgLuc/NANOLUC-based polypeptide and peptide sequences in U.S. patent Ser. No. 16/439,565 differ from the corresponding sequences in NANOLUC and wild-type native OgLuc). In some embodiments, first and second components of the luminescent reporters in the systems and methods herein comprise or are based on the components of these systems.

In some embodiments, peptide and polypeptide components are provided (e.g., as the first and second components of the luminescent reporter of embodiments herein) for the assembly of a bioluminescent complex capable of generating bioluminescence in the presence of an appropriate substrate (e.g., a coelenterazine or a coelenterazine analog (e.g., furimazine, fluorofurimazine, etc.). In some embodiments, complementary polypeptide(s) and peptide(s) collectively span the length (or >75% of the length, >80% of the length, >85% of the length, >90% of the length, >95% of the length, or more) of a luciferase base sequence (or collectively comprise at least 40% sequence identity to a luciferase base sequence (e.g., >40%, >45%, >50%, >55%, >60%, >65%, >70%, >75%>80%, >85%, >90%, >95%, or more). In some embodiments, complementary polypeptide(s) and peptide(s) are separate molecules that each correspond to a portion of a luciferase base sequence. Through structural complementarity, they assemble to form a bioluminescent complex. Suitable base sequences may include SEQ ID NO: 1, or the sequences of any of the full-length luciferases described herein or understood in the field. In some embodiments, the bioluminescent complex comprises the NANOBIT or NANOTRIP systems (Promega; Madison, WI). In some embodiments, the peptide and/or polypeptide components of a bioluminescent complex collectively comprise at least 60% sequence identity (e.g., >60%, >65%, >70%, >75%, >80%, >85%, >90%, >95%, >99%) with SEQ ID NO: 1. In some embodiments, the peptide and/or polypeptide components of the bioluminescent complex comprise LGBIT (SEQ ID NO: 2), SMBIT (SEQ ID NO: 3), LGTRIP (SEQ ID NO: 4), and/or SMTRIP9 (SEQ ID NO: 5), SMTRIP10 (SEQ ID NO: 6), and/or HIBIT (SEQ ID NO: 7). In some embodiments, the peptide and/or polypeptide components (e.g., first and second components of the luminescent reporter) of the bioluminescent complex comprise at least 60% sequence identity (e.g., >60%, >65%, >70%, >75%, >80%, >85%, >90%, >95%, >99%) with LGBIT (SEQ ID NO: 2), SMBIT (SEQ ID NO: 3), LGTRIP (SEQ ID NO: 4), SMTRIP9 (SEQ ID NO: 5) SMTRIP10 (SEQ ID NO: 6), and/or HIBIT (SEQ ID NO: 7).

In some embodiments, a first component of a luminescent reporter comprises at least 60% sequence identity (e.g., >60%, >65%, >70%, >75%, >80%, >85%, >90%, >95%, >99%, 100%) with LGBIT and a second component of a luminescent reporter comprises at least 60% sequence identity (e.g., >60%, >65%, >70%, >75%, >80%, >85%, >90%, >95%, >99%, 100%) with SMBIT. In some embodiments, a first component of a luminescent reporter comprises at least 60% sequence identity (e.g., >60%, >65%, >70%, >75%, >80%, >85%, >90%, >95%, >99%, 100%) with SMBIT and a second component of a luminescent reporter comprises at least 60% sequence identity (e.g., >60%, >65%, >70%, >75%, >80%, >85%, >90%, >95%, >99%, 100%) with LGBIT.

In some embodiments, a first component of a luminescent reporter comprises at least 60% sequence identity (e.g., >60%, >65%, >70%, >75%, >80%, >85%, >90%, >95%, >99%, 100%) with LGTRIP and a second component of a luminescent reporter comprises at least 60% sequence identity (e.g., >60%, >65%, >70%, >75%, >80%, >85%, >90%, >95%, >99%, 100%) with a fusion of SMTRIP9 and SMTRIP10.

In some embodiments, a first component of a luminescent reporter comprises at least 60% sequence identity (e.g., >60%, >65%, >70%, >75%, >80%, >85%, >90%, >95%, >99%, 100%) with LGTRIP, a second component of a luminescent reporter comprises at least 60% sequence identity (e.g., >60%, >65%, >70%, >75%, >80%, >85%, >90%, >95%, >99%, 100%) with a fusion of SMTRIP9, and a third component of a luminescent reporter (e.g., added as a separate detection reagent (e.g., with the substrate)) comprises at least 60% sequence identity (e.g., >60%, >65%, >70%, >75%, >80%, >85%, >90%, >95%, >99%, 100%) with a fusion of SMTRIP10.

In some embodiments, any of the aforementioned components of bioluminescent complexes are linked (e.g., fused, chemically linked, linked via primary/secondary antibodies, tethered, etc.) to one or more other elements (e.g., target protein, cysteine-reactive moiety, etc.) of the systems described herein (e.g., fused to a HALOTAG protein, an antibody, etc.).

In embodiments in which a luminescent reporter functions by structural complementation to form a bioluminescent complex, suitable substrates are provided that emit light upon interaction with the assembled bioluminescent complexes described herein. Suitable substrates for the bioluminescent complexes herein will be understood. Any of the bioluminescent substrates described above (for bioluminescent protein donors) may find use with appropriate bioluminescent complexes. Pairing of appropriate bioluminescent complexes with bioluminescent substrates is understood in the field. In particular embodiments, a bioluminescent complex utilizes an imidazopyrazine substrate, such as coelenterazine, furimazine, or fluorofurimazine (U.S. application Ser. No. 16/548,214; incorporated by reference in its entirety). In some embodiments, systems and methods herein comprise a structural-complementation-based bioluminescent reporter (e.g., first and second components) and an imidazopyrazine substrate such as native coelenterazine, furimazine, hydrofurimazine, fluorofurimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); and U.S. Pat. No. 8,669,103; the disclosures of which are incorporated by reference herein in their entireties. In some embodiments, the substrate and bioluminescent complex emit light upon interaction of the substrate with the bioluminescent complex assembled from the first and second components. In some embodiments, the complex/substrate emits light in the visible light spectrum (e.g., about 400 to about 700 nm (e.g., 400 nm, 425 nm, 450 nm, 475 nm, 500 nm, 525 nm, 550 nm, 575 nm, 600 nm, 625 nm, 650 nm, 675 nm, 700 nm, or ranges therebetween). In some embodiments, the substrate emits light of a wavelength between 400 and 500 nm (e.g., 400 nm, 410 nm, 420 nm, 430 nm, 440 nm, 450 nm, 460 nm, 470 nm, 480 nm, 490 nm, 500 nm, or ranges therebetween).

There are various characteristics of the bioluminescent complexes that find use in embodiments herein that may provide advantages in certain applications. For example, a bioluminescent complex (e.g., a complex formed upon complementation of first and second complementary components of a bioluminescent complex (e.g., SmBiT and LgBiT)) only generates light upon complementation of its component peptide/polypeptides; therefore, directly or indirectly conjugating (e.g., fusing, tethering, etc.) one or more components of the bioluminescent complex to other components of the system (e.g., cysteine-reactive moiety, target protein, etc.) ensures that the bioluminescent complex will form, only when the other components are brought into proximity of each other (e.g., binding of the cysteine-reactive moiety of the tracer to the target protein). Tethering of two other components of the system to separate components of the bioluminescent complex ensures the proximity of those components upon light generation by the complex. In some embodiments, the use of a bioluminescent complex, due to the requirement that two components come together to form the complex, provides enhanced spatiotemporal resolution through conditional activation at a specific site.

In some embodiments a component of the bioluminescent complex is inserted in an internal position within the target protein or another element (e.g., HALOTAG) used to tether the components to the target protein.

### III. Cysteine-Reactive Tracer

In some embodiments, methods and systems herein comprise a cysteine-reactive tracer. In some embodiments, unlike the test agents that find use in the embodiments herein, the tracer is not specific for a target site and/or target protein. Instead, the cysteine-reactive tracers are capable of binding non-specifically to thiols on cysteine residues of proteins. Therefore, when the tracer is added to a sample comprising various proteins (e.g., a cell or cell lysate, a biochemical mixture, etc.), it non-specifically binds to cysteines on the proteins therein, including but not limited to a target cysteine at the target site of a target protein.

### A. Cysteine-Reactive Moiety

In some embodiments, the cysteine-reactive tracer comprises a cysteine-reactive moiety linked a component (the “second” component) of a luminescent reporter (the “first” component being linked to the target protein).

In some embodiments, a cysteine-reactive moiety of a tracer is an electrophilic group. In some embodiments, the cysteine-reactive moiety is selected from a maleimide moiety, an acrylamide moiety, a chloroacetamide moiety, a geminal dicyanocyclopropane carboxamide moiety, a bromo-dihydroisoxazole carboxamide moiety, and an azetidinyl oxadiazole moiety. In some embodiments, a cysteine-reactive test agent comprises a structure of:

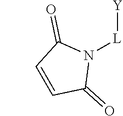

(tracer with maleimide cysteine-reactive moiety);

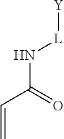

(tracer with acrylamide cysteine-reactive moiety);

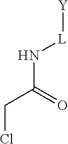

(tracer with chloroacetamide cysteine-reactive moiety);

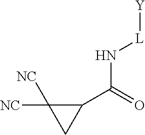

(tracer with geminal dicyanocyclopropane carboxamide cysteine-reactive moiety);

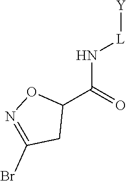

(tracer with bromo-dihydroisoxazole carboxamide cysteine-reactive moiety); and

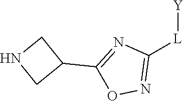

(test agent with azetidinyl oxadiazole cysteine-reactive moiety); wherein L is a linker, and Y is a component of the luminescent reporter (e.g., an acceptor fluorophore, a components of a bioluminescent complex, etc.).

### B. Cysteine-Reactive Fluorescent Tracer

In some embodiments, methods and systems herein comprise a cysteine-reactive fluorescent tracer. In some embodiments, the cysteine-reactive fluorescent tracer comprises a cysteine-reactive moiety and a fluorescent moiety (e.g., acceptor fluorophore). In some embodiments, a tracer herein comprises a fluorescent moiety (e.g., linked to a cysteine-reactive moiety). The fluorophore may be a small molecule fluorophore capable of emitting a detectable fluorescent signal. In some embodiments, the fluorophore serves as an energy transfer acceptor (e.g., a BRET acceptor).

In some embodiments, the fluorophore is a small molecule (e.g., molecular weight less than 3,000 daltons, <2,500 daltons, <2,000 daltons, <1,500 daltons, <1,000 daltons, <900 daltons, <800 daltons, <700 daltons, <600 daltons).

Suitable fluorophores for use as fluorescent moieties herein include, but are not limited to stilbazolium derivatives (Marquesa et al. Mechanism-Based Strategy for Optimizing HALOTAG Protein Labeling. ChemRxiv. Cambridge: Cambridge Open Engage; 2021; incorporated by reference in its entirety), xanthene derivatives (e.g., fluoresceins, rhodamines, rhodols, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), CF dye (Biotium), BODIPY (Invitrogen), ALEXA FLUOR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), etc.

In some embodiments, the fluorophore is a rhodol or rhodamine dye (Beija et al. Chem. Soc. Rev., 2009, 38, 2410-2433; incorporated by reference in its entirety) or a variant or derivative thereof. In some embodiments, the rhodol or rhodamine dye is selected from:

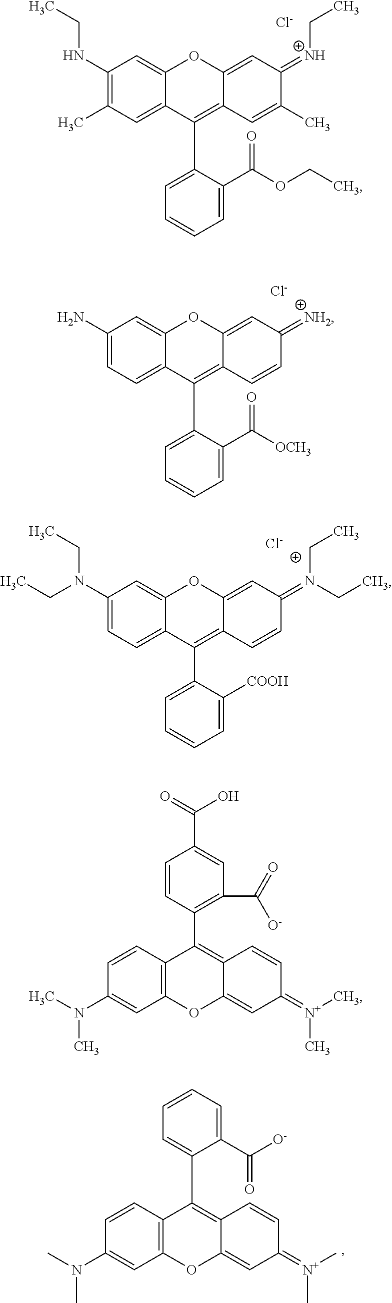

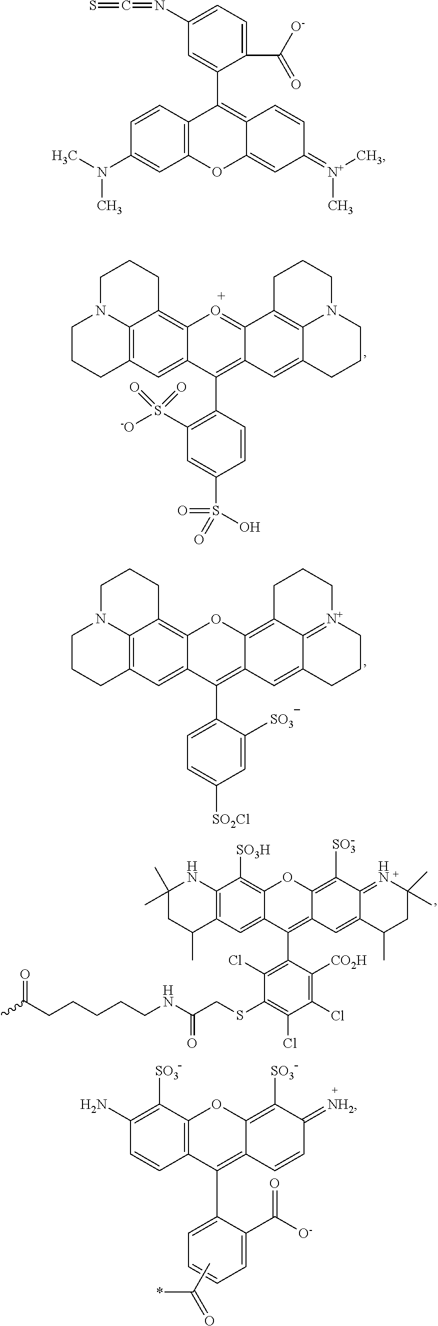

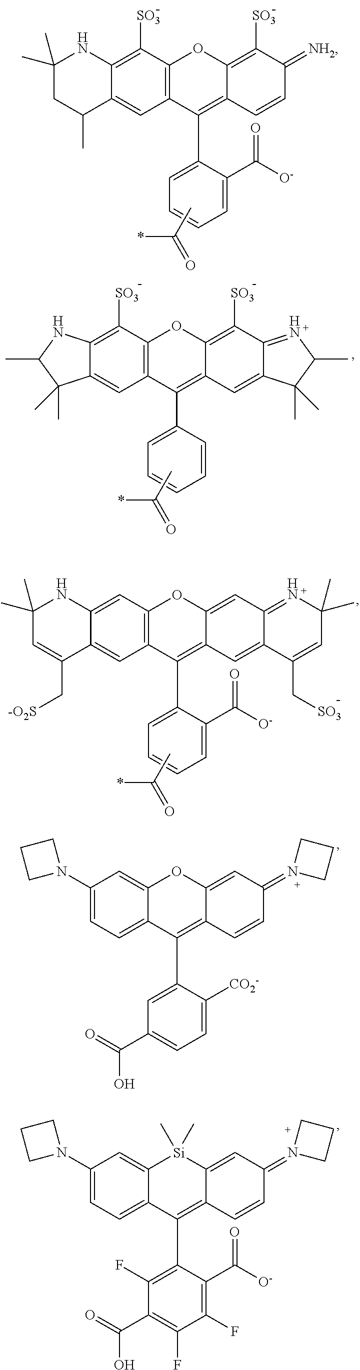

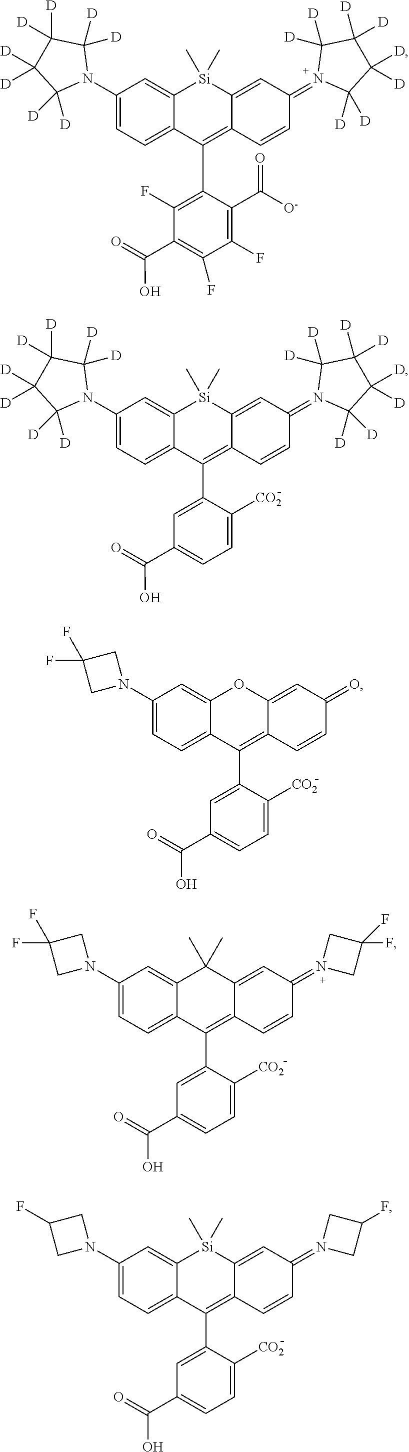

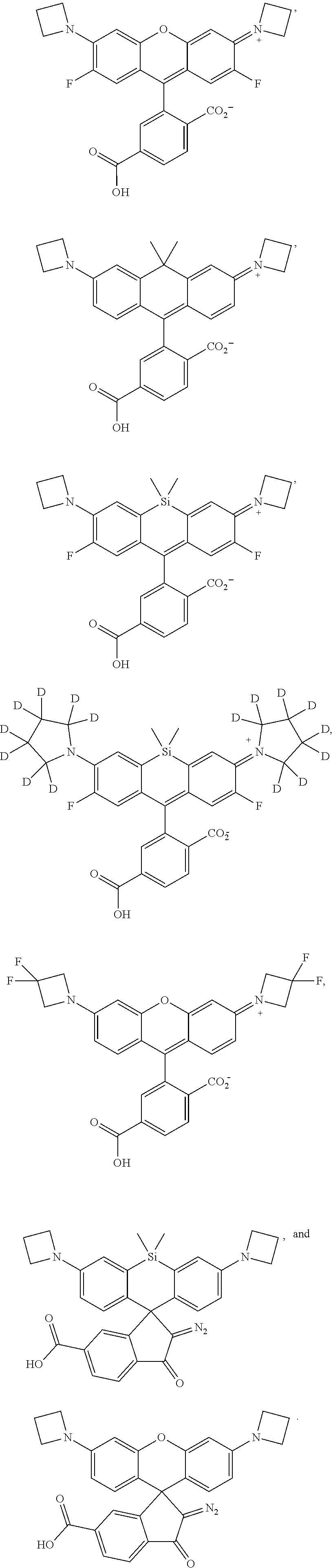

In some embodiments, the fluorophore is a rhodamine.

In some embodiments, a fluorescent moiety is a fluorogenic functional group. A fluorogenic functional group is one that produces and enhanced fluorescent signal upon binding of a molecular entity of which it is a component to a target (e.g., binding of a fluorogenic tracer to a target protein). By producing significantly increased fluorescence (e.g., 10×, 31×, 50×, 100×, 310×, 500×, 100×, or more) upon target engagement, background signal is reduced. Exemplary fluorogenic dyes for use in embodiments herein include the JANELIA FLUOR family of fluorophores, such as:

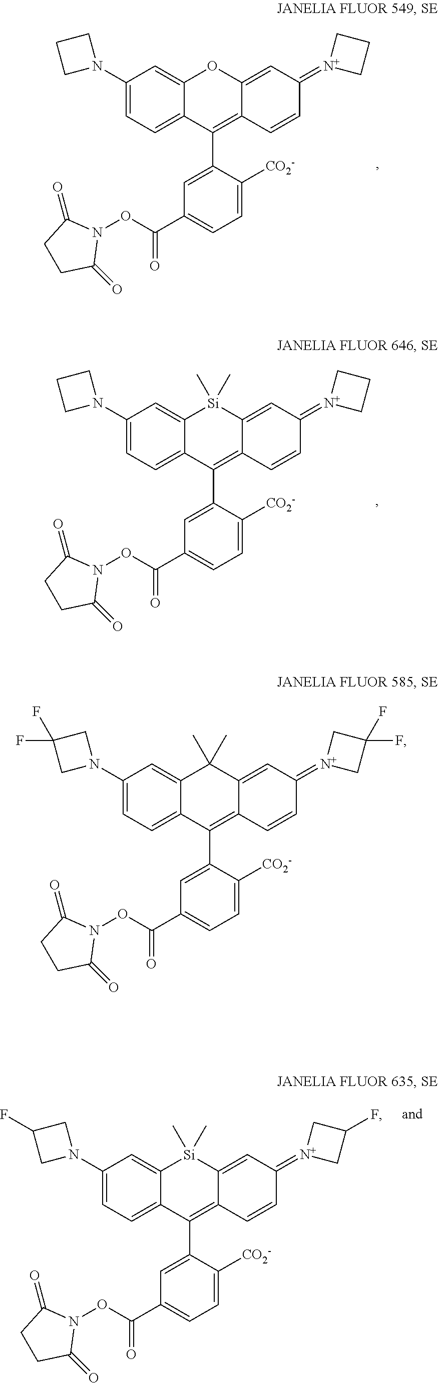

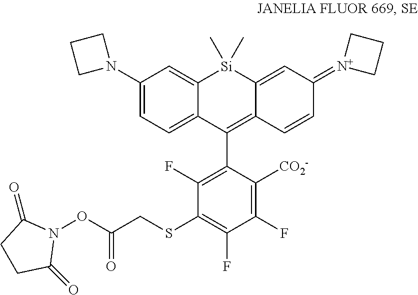

(see, e.g., U.S. Pat. Nos. 9,933,417; 10,018,624; 10,161,932; and 10,495,632; each of which is incorporated by reference in their entireties). The use and design of fluorogenic functional groups, dyes, probes, and substrates is described in, for example Grimm et al. Nat Methods. 3117 October; 14(10):987-994.; Wang et al. Nat Chem. 3120 Feb; 12(2):165-172; incorporated by reference in their entireties.

In some embodiments, a cysteine-reactive moiety and fluorescent moiety are directly covalently bonded. In other embodiments, the cysteine-reactive moiety and fluorescent moiety are tethered to each other by a linker (L). Exemplary chemical linkers may comprise functional groups selected alkylene, arylene, —O, —NH—, carbamate, and —C(O)— groups. For example, linkers may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)).

In some embodiments, linker L of a cysteine reactive fluorescent tracer comprises —(CH₂)ₓNHC(O)(CH₂)_{y}—, wherein x and y are independently selected from 0-6.

In some embodiments, linker L of a cysteine reactive fluorescent tracer comprises a linker selected from:

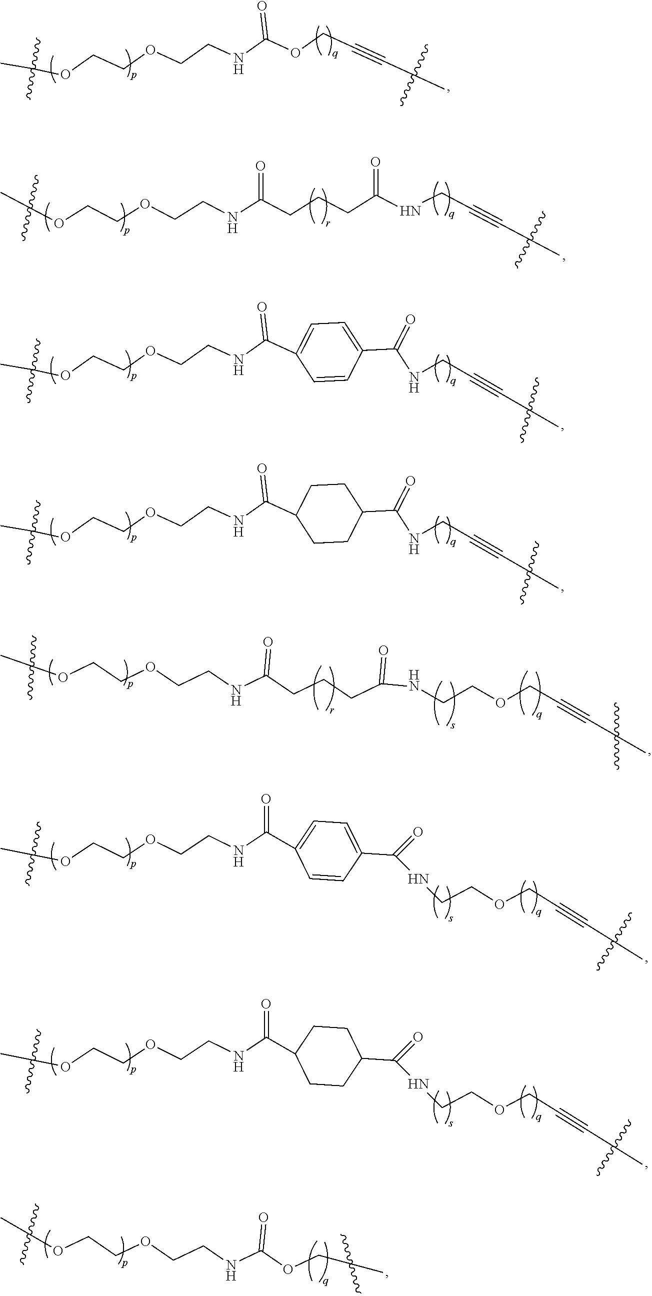

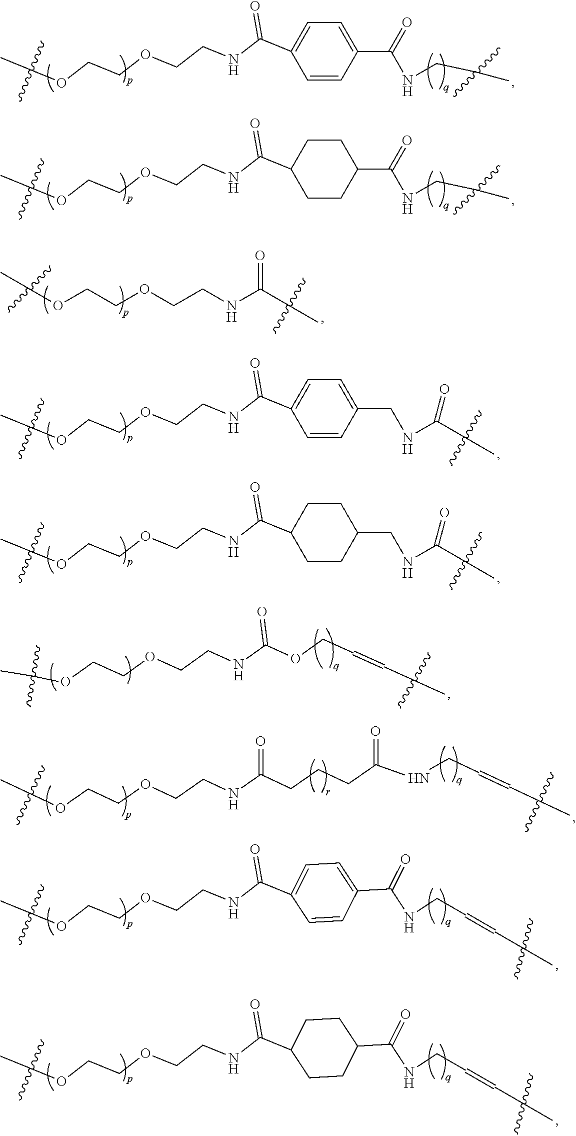

where p, q, r, and s are each independently 1-6. Other linkers suitable for tethering a cysteine-reactive moiety to a fluorescent moiety are understood in the field.

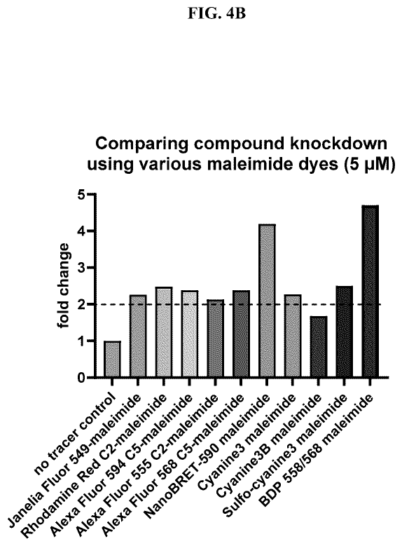

Exemplary tracers for use in embodiments herein include the panel of maleimide-based tracers depicted in FIG. 4A. In some embodiments, suitable tracers for use in embodiments herein include the tracers of FIG. 4A with alternative linkers described herein, alternative cysteine binding moieties described herein, and/or alternative fluorescent moieties described herein. In some embodiments, a tracer is selected from:

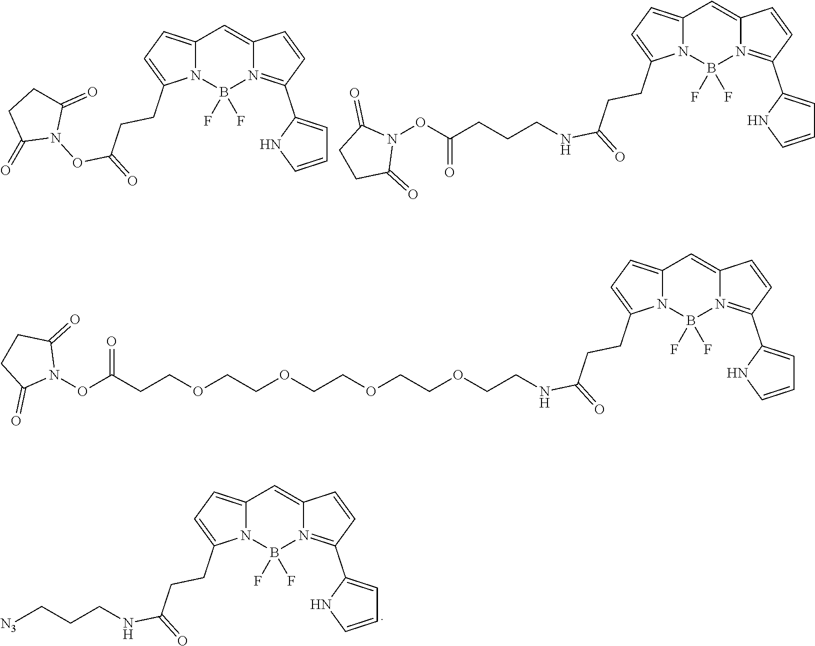

In some embodiments an exemplary tracer for use in embodiments herein is of the structure:

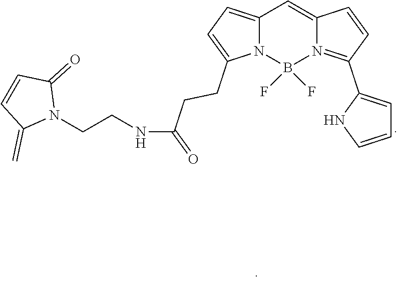

Any other suitable combinations of cysteine-reactive moiety, linker, and fluorophore may find use herein.

### C. Cysteine-Reactive, Structurally-Complementary Tracer

In some embodiments, methods and systems herein comprise a cysteine-reactive structurally-complementary tracer. In some embodiments, the cysteine-reactive structurally-complementary tracer comprises a cysteine-reactive moiety and a component of a bioluminescent complex (e.g., the other component being tethered to the target protein). In some embodiments, a tracer herein comprises a component of a bioluminescent complex (e.g., linked to a cysteine-reactive moiety). The component of a bioluminescent complex may be a peptide or polypeptide that is capable of interesting (e.g., binding) to another component of the bioluminescent complex to form an active bioluminescent complex when the elements to which they are attached (e.g., target protein and cysteine-reactive moiety) are brought into proximity of each other (e.g., upon binding the cysteine-reactive moiety to a thiol on the target protein). In some embodiments, upon formation form the separate components, the bioluminescent complex is capable of emitting light (e.g., at an emission wavelength) in the presence of an appropriate substrate.

### IV. BRET Reporters

In some embodiments, the two portions of the luminescent reporter, (1) the bioluminescent protein tethered to the target protein and (2) the acceptor fluorophore of the tracer, are selected as a pair to allow for bioluminescence resonance energy transfer (BRET from the bioluminescent protein to the acceptor fluorophore). Appropriate BRET pairs include any bioluminescent protein with an emission spectrum that overlaps to a significant enough degree with the excitation spectrum of the acceptor fluorophore to provide excitation of the fluorophore by the bioluminescent protein when the fluorophore and bioluminescent protein are brought within sufficient proximity of each other (e.g., within 30-80 Å, depending on the degree of spectral overlap), such as by binding of a tracer comprising the fluorophore to a target protein tethered to the bioluminescent protein.

### V. FRET Reporters

In some embodiments, the two portions of the luminescent reporter, (1) the donor fluorophore tethered to the target protein and (2) the acceptor fluorophore of the tracer, are selected as a pair to allow for fluorescence resonance energy transfer (e.g., FRET from the donor fluorophore to the acceptor fluorophore). Appropriate FRET pairs include any fluorescent protein or donor fluorophore with an emission spectrum that overlaps to a significant enough degree with the excitation spectrum of the acceptor fluorophore of the tracer to provide excitation of the acceptor fluorophore by the fluorescent protein or donor fluorophore, when the tracer and target protein are brought within sufficient proximity of each other (e.g., within 30-80 Å, depending on the degree of spectral overlap), such as by binding of a tracer comprising the acceptor fluorophore to a target protein tethered to the fluorescent protein or donor fluorophore.

### VI. Structural Complementation Reporters

In some embodiments, the two portions of the luminescent reporter, (1) the first component of the bioluminescent complex tethered to the target protein and (2) the first component of the bioluminescent complex as part of the tracer, are selected as a pair to allow for structural complementation between the two components to produce a bioluminescent complex capable of emitting a bioluminescent signal. Appropriate complementary pairs include any peptide/polypeptide components, such as SmBiT and LgBiT, that are capable of coming together to produce a bioluminescent complex.

### VII. Conjugation

The systems and methods herein comprise various elements/components tethered together, such as (1) a target protein to a first component of a luminescent reporter, (2) a cysteine-reactive moiety to a targeting moiety, and (3) a cysteine-reactive moiety to a second component of a luminescent reporter. The components may be tethered by any suitable structure or mechanism.

In embodiments in which both of the linked elements/components are proteins, polypeptides, and/or peptides, the elements/components may be fused together as a single fusion polypeptide (or fusion protein). In some embodiments, such a construct may be expressed as a single fusion protein and encoded on a single nucleic acid or vector. A construct with two elements/components within a fusion protein can be expressed within a cell (e.g., during a method herein). The two peptide/polypeptide elements can be fused directly to each other, or the fusion may comprise a linker peptide/polypeptide sequence. The linker may be 1-100 amino acids in length (e.g., 1, 2, 3, 4, 5, 10, 15, 20, 25, 30, 40, 50, 60, 70, 80, 90, 100, or ranges therebetween) or longer. The linker may be of any suitable sequence that allows the tethered components to properly function and to be properly aligned for the methods herein.

In some embodiments, two elements/components are covalently tethered together by a linker. A linker tethering two elements/components (e.g., a cysteine-reactive moiety to a targeting moiety or component of a luminescent reporter) may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—wherein z1, z2, z3, and z4 are each independently selected form 0, 1, 2, 3, 4, 5, and 6. For example, in some embodiments, the linker has a formula selected from:

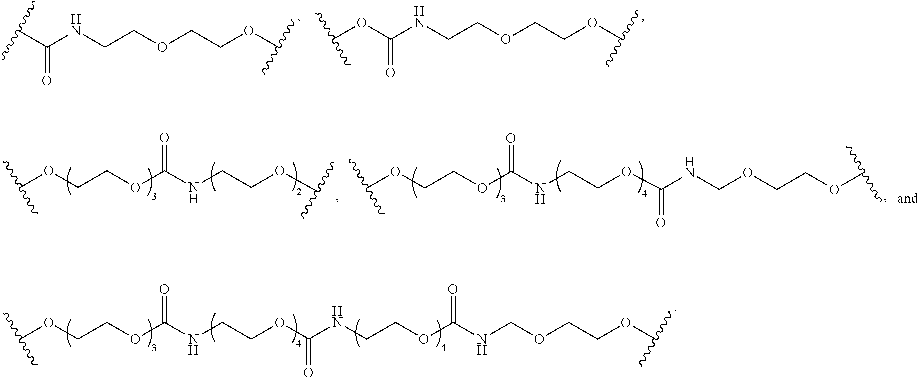

In some embodiments, two elements/components of a system herein are tethered together via the interaction of an affinity molecule (e.g., antibody, antibody fragment, streptavidin, etc.) and an affinity tag (e.g., epitope, biotin, etc.). In some embodiments, the affinity molecule (e.g., antibody, antibody fragment, streptavidin, etc.) is fused to one component (e.g., target protein, component of a bioluminescent complex, luciferase, fluorescent protein, etc.), and the affinity tag (e.g., epitope, biotin, etc.) is fused to the second element/component. Interaction of the affinity molecule and tag in a system herein results in tethering of the two elements/components.

In some embodiments, two elements/components of a system herein are tethered together via the interaction of a secondary affinity molecule (e.g., antibody, antibody fragment, etc.) with a primary affinity molecule (e.g., antibody, antibody fragment, streptavidin, etc.) that is capable of binding to a component of the system or an affinity tag (e.g., epitope, biotin, etc.) linked thereto. In some embodiments, a first component of the system (e.g., component of the bioluminescent complex, bioluminescent protein, etc.) becomes tethered to a second component of the system (e.g., target protein) when a secondary affinity molecule linked to the first component binds to a primary affinity molecule bound to the target protein (or an affinity tag thereon).

In some embodiments, two elements/components of a system herein are tethered together via the interaction of a capture agent (e.g., HALOTAG) and a capture ligand (e.g., chloroalkane molecule). In some embodiments, the capture agent (e.g., HALOTAG) is fused to one component (e.g., target protein, component of a bioluminescent complex, luciferase, fluorescent protein, etc.) and the capture ligand (e.g., chloroalkane molecule) is chemically linked to the second element/component. Covalent binding of the capture agent and capture ligand in a system herein results in tethering of the two components.

An exemplary capture agent/ligand system that finds use in tethering elements/components of the systems herein is a modified dehalogenase (e.g., HALOTAG) and a haloalkyl ligand (e.g., HALOTAG ligand). HALOTAG is a 297-residue self-labeling polypeptide (33 kDa) derived from a bacterial hydrolase (dehalogenase) enzyme, which has been modified to covalently bind to its ligand, a haloalkane moiety. The HALOTAG ligand can be linked to a moiety/element/components and the HALOTAG polypeptide can be fused to peptide/polypeptide components; covalent binding of the ligand by HALOTAG results in covalent tethering of the moiety/element/components attached thereto.

The HALOTAG polypeptide is a modified dehalogenase with a genetically modified active site, which specifically binds to the haloalkane ligand chloroalkane linker with an enhanced and increased rate of ligand binding (Pries et al. The Journal of Biological Chemistry. 270(18):10405-11; incorporated by reference in its entirety). The reaction that forms the bond between the protein tag and chloroalkane linker is fast and essentially irreversible under physiological conditions (Waugh DS (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety).

HALOTAG fusion proteins can be expressed using standard recombinant protein expression techniques (Adams et al. (May 2002) Journal of the American Chemical Society. 124(21):6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5):571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen DB (April 2004). Current Opinion in Chemical Biology. 8(2):150-9; incorporated by reference in its entirety).

In some embodiments, a ligand for a modified dehalogenase is provided herein. In some embodiments, the ligand is of the structure R-linker-A-X, wherein R the target protein (protein of interest), wherein A is (CH₂)₂₋₁₂, wherein X is a halogen, and wherein the linker is a linker moiety capable of tethering R to A-X. In some embodiments, the linker is a multiatom straight or branched chain including C, N, S, or O, or a group that comprises one or more rings, e.g., saturated or unsaturated rings, such as one or more aryl rings, heteroaryl rings, or any combination thereof. In some embodiments, the linker comprises a combination of —O(CH₂)₂— —(CH₂)O—, —CH₂—, —NHC(O)O—, —OC(O)NH—, NHC(O)—, and —C(O)NH—. In some embodiments, the linker is 5 to 50 (e.g., 5, 10, 15, 20, 25, 30, 35, 40, 45, 50, or ranges therebetween) atoms in length. In some embodiments, the length of the linker for tethering the fluorophore allows for optimization of proximity and geometry (e.g., for efficient energy transfer).

Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

In some embodiments, two elements/components of a system herein are tethered together via a chemical linkage. Chemical linkage may be by any suitable conjugation method, such as, click chemistry, thiol-maleimide linkage, cysteine-maleimide-cysteine conjugation, etc. U.S. Pub No. 2020/0166460 (incorporated by reference in its entirety) describes peptides labeled with sulfo n-hydroxysuccinimidyl ester (sulfo-SE) moieties and the use of such reactive labeled peptides for the labeling of proteins via covalent conjugation to primary amines on the protein. Such chemistry can be employed herein to covalently link peptides (e.g., SmBiT) or small molecules (e.g., chloroalkane, cysteine-reactive moiety, targeting moiety, fluorophore, etc.) to primary amines on proteins (e.g., target protein, luciferase, LgBiT, etc.) within the systems herein.

### VIII. Kits, Systems, and Methods

In some embodiments, provided herein are compositions, kits, cells, and systems comprising one or more (e.g., all) of: target protein tethered to a first component of a luminescent reporter; a cysteine-reactive tracer comprising a second component of a luminescent reporter tethered to a cysteine-reactive moiety; a cysteine-reactive test agent (e.g., comprising a targeting moiety and a cysteine-reactive moiety). In some embodiments, kits, cells, and systems further comprise a substrate for any bioluminescent components or complexes therein.

In some embodiments provided herein is a cell, tissue, organism, cell-free expression system, cell lysate, or other sample comprising a nucleic acid (e.g., DNA or RNA) encoding any fusions, bioluminescent proteins, components of bioluminescent complexed, fluorescent proteins, target proteins, capture agents, antibodies, etc. herein. In some embodiments, the nucleic acid is incorporated into the genome of the cell, tissue, organism, etc. In other embodiments, the nucleic acid is present within an exogenous vector. In some embodiments provided herein are cells, tissues, organisms, cell-free expression systems, cell lysates, or other samples comprising the elements/components of the system described herein. In some embodiments, methods are provided for incorporating a nucleic acid (e.g., DNA or RNA) encoding a elements/components of the system described herein into a cell, tissue, organism, cell-free expression system, cell lysate, or other sample. In some embodiments, methods are provided for expressing a fusion, target protein, bioluminescent protein, fluorescent protein, component of a bioluminescent complex, etc. from a nucleic acid (e.g., DNA or RNA) within a cell, tissue, organism, cell-free expression system, cell lysate, or other sample.

In some embodiments, provided herein is a composition, kit, biochemical reagent, or system comprising one or more of a cysteine-reactive tracer comprising a component of a luminescent reporter tethered to a cysteine-reactive moiety; a cysteine-reactive test agent (e.g., comprising a targeting moiety and a cysteine-reactive moiety); and a substrate for the bioluminescent protein. In some embodiments, provided herein is a composition, kit, biochemical reagent, or system comprising a cysteine-reactive tracer comprising a component of a luminescent reporter tethered to a cysteine-reactive moiety and a substrate for the bioluminescent protein.

In some embodiments provided herein are methods comprising administering a cysteine-reactive test agent (e.g., comprising a targeting moiety and a cysteine-reactive moiety) to a cell, tissue, organism, cell-free expression system, cell lysate, or other sample comprising a target protein tethered to a first component of a luminescent reporter.

In some embodiments, provided herein are methods comprising administering a cysteine-reactive tracer comprising a fluorophore tethered to a cysteine-reactive moiety and/or a substrate for a bioluminescent protein or complex to a cell, tissue, organism, cell-free expression system, cell lysate, or other sample comprising a cysteine-reactive test agent (e.g., comprising a targeting moiety and a cysteine-reactive moiety) and a target protein tethered to a first component of a luminescent reporter.

In some embodiments, methods comprise detecting and/or monitoring over time a fluorescent signal resulting from BRET from a bioluminescent protein (e.g., fused to a target protein) to a fluorophore (e.g., tethered to a cysteine-reactive moiety) or FRET from a fluorescent tracer fused to a target protein to a fluorophore tethered to a cysteine-reactive moiety. In some embodiments, methods comprise inversely correlating the BRET signal to the amount of covalent binding of a cysteine-reactive test agent (e.g., comprising a targeting moiety and a cysteine-reactive moiety) to a target cysteine at a target site on a target protein.

In some embodiments, methods herein are performed within a cell. In some embodiments, the components of the assays herein are either expressed within the cell or added exogenously and allowed to enter the intact cell. In some embodiments, methods herein are performed within a cell. In such embodiments, assays/methods may comprise a step of cell lysis. In some embodiments, a portion of the assays described herein is performed within a cell (e.g., expressing the target protein, interaction of the test agent with the target protein, etc.) and a portion of the assay is performed in a cell lysate generated from the cell (e.g., binding of tracer). Accordingly, in some embodiments, method herein provide a step of lysis a cell to produce a cell lysate, and compositions/techniques for cell lysis are within the scope herein. In some embodiments, agents for cell lysis may be provided with the kits described herein. Suitable agents for use with the assays described herein can be selected. In some embodiments, agents for cell lysis (and use in the methods herein or inclusion in systems/kits herein) include chemical agents, such as detergents (e.g., Triton X-100, NP-40, CHAPS, SDS, sodium deoxycholate, digitonin, etc.), hypotonic solution, chaotropic agents (e.g., Urea, guanidine hydrochloride, etc.), etc. In some embodiments, agents for cell lysis (and use in the methods herein or inclusion in systems/kits herein) include enzymatic agents, such as lysozyme, zymolase, lyticase, proteases, nucleases, etc. In some embodiments, cells are lysed using physical/mechanical techniques, such as sonication, homogenization, high pressure (e.g., French press), bead beating, freeze/thaw, etc. In some embodiments, cells are lysed using osmotic shock, heat, lysis buffers, or combinations of the above. Methods, composition, and kits for cell lysis are provided herein.

### Experimental

### Example 1

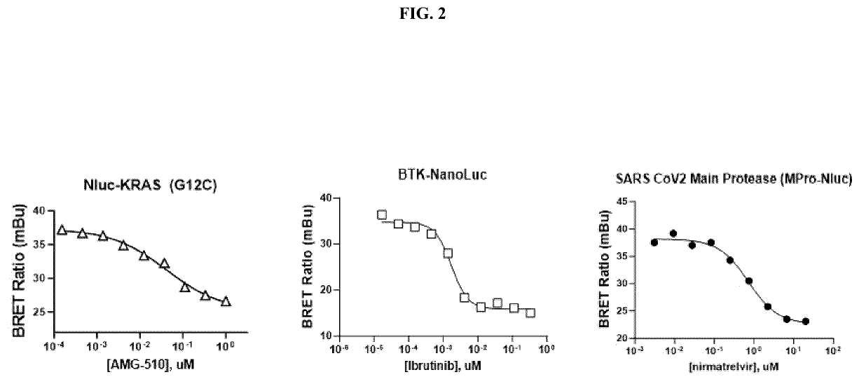

HEK293 cells were transfected with plasmids encoding NANOLUC-tagged proteins (KRAS (G12C), BTK, and SARS COV2 MPro; 5 ng/well), plated in 96-well plates (Corning 3917) at 20,000 cells/well, and allowed to express overnight at 37° C. After 20 h, the cells were treated with known positive control test compounds for 1 hour. After 1h, the cells were treated with 5 micromolar maleimide NanoBRET-590 and incubated for 1 additional hour. NanoBRET® TE substrate/inhibitor reagent was added to cells to a 1× concentration, and BRET was recorded on a GloMax® Discover luminometer (FIG. 2).

### Example 2

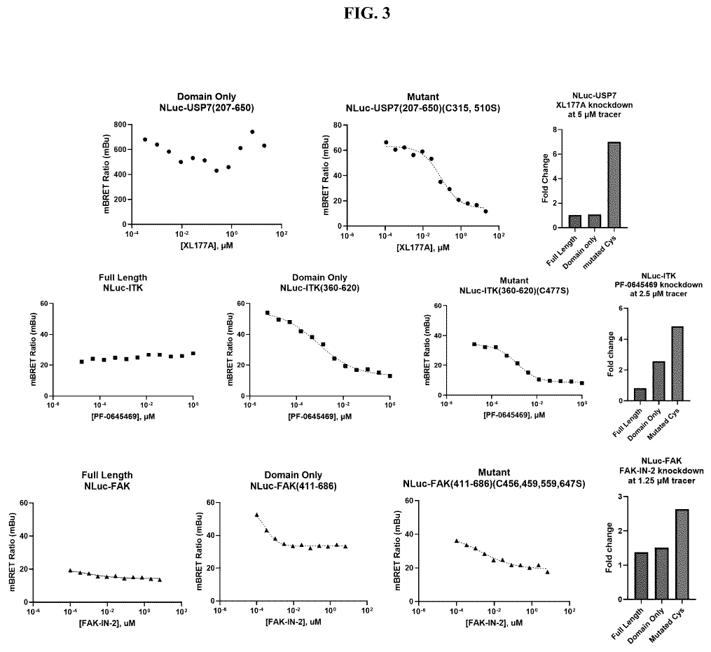

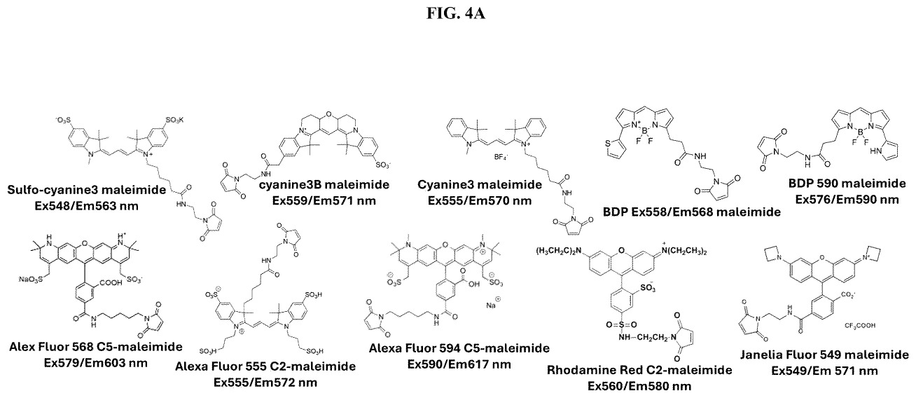

HEK293 cells were transfected with plasmids (5 ng/well) encoding for either: 1) NANOLUC-tagged full length proteins (FAK, ITK, USP7), 2) NANOLUC-tagged domain-only proteins (FAK(411-686), ITK(360-620), USP7(207-650)), or 3) NANOLUC-tagged domain-only mutated proteins, where solvent exposed cysteines were mutated to serine (FAK(411-686)(C456,459,559, 647S) and USP7(207-650)(C315,510S)). Transfected cells were placed in a cell culture flask and allowed to incubate overnight at 37° C. for 16-20 hrs in growth media. The next morning, cells were harvested and plated into wells of 96-well plates (Corning 3917) at 20,000 cells/well in a phenol red free buffered media at biological pH. Cells were treated with known positive control test compounds at dose response or vehicle for 2 hrs. After 2 hrs, the cells were treated with varying optimal concentrations of maleimide NanoBRET-590 for each target, immediately lysed with 50 μg/mL of digitonin, and incubated for 30 min at room temperature in the dark. NanoBRET® TE substrate reagent (Promega) was added to cells to a 1× concentration, and BRET was recorded on a GloMax® Discover Luminometer (FIG. 3A).

HEK293 cells were transfected with plasmids (5 ng/well) encoding for either: 1) NANOLUC-tagged full length proteins (FAK, ITK, USP7), 2) NANOLUC-tagged domain-only proteins (FAK(411-686), ITK(360-620), USP7(207-650)), or 3) NANOLUC-tagged domain-only mutated proteins, where solvent exposed cysteines were mutated to serine (FAK(411-686)(C456,459,559, 647S), USP7(207-650)(C315,510S)). Transfected cells were placed in a cell culture flask and allowed to incubate overnight at 37° C. for 16-20 hrs in growth media. The next morning, cells were harvested and plated into wells of 96-well plates (Corning 3917) at 20,000 cells/well in a phenol red free buffered media at biological pH. Cells were treated with 20 μM of a known positive control test compounds or vehicle for 2 hrs. After 2 hrs, the cells were treated 20 with varying optimal concentrations of maleimide NanoBRET-590 for each target, immediately lysed with 50 μg/mL of digitonin, and incubated for 30 min at room temperature in the dark. NanoBRET® TE substrate reagent was added to cells to a 1× concentration and BRET was recorded on a GloMax® Discover Luminometer. Compound knockdown fold-change was calculated by dividing the no test compound wells by the 20 μM test compound well (FIG. 3B).

Data in FIG. 3 demonstrates the assay can be optimized through molecular biology efforts, either truncating the target protein or mutating solvent exposed cysteines. Additionally, mutation of the solvent exposed cysteines to serine enabled mapping of the relevant cysteine participating in the target engagement event.

### Example 3

HEK293 cells were transfected with plasmids (5 ng/well) encoding NANOLUC-tagged BTK and placed in a cell culture flask. Transfected cells were incubated overnight at 37° C. for 16-20 hrs in growth media. The next morning, cells were harvested and plated into wells of 96-well plates (Corning 3917) at 20,000 cells/well in a phenol red free buffered media at biological pH. Cells were treated with a known positive control test compound or vehicle for 2 hrs. After 2 hrs, the cells were treated with a maleimide dye in dose response (5 μM, 4 pt, 1:2), immediately lysed with 50 μg/mL of digitonin, and incubated for 30 min at room temperature in the dark. NanoBRET® TE substrate reagent was added to cells to a 1× concentration and BRET was recorded on a GloMax® Discover Luminometer (FIG. 4). Compound knockdown fold change was calculated to determine the best performing maleimide dye.

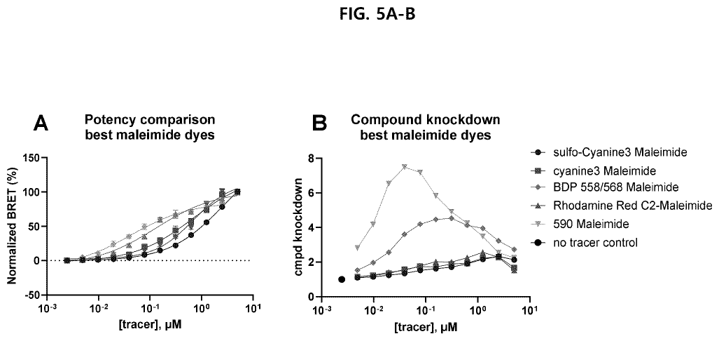

The best performing maleimide dyes were further evaluated in a 11 pt dose response (5 μM, 1:2). EC₅₀ curves were normalized to control to better compare reactivity (FIG. 5A). Compound knockdown fold-change was calculated by dividing the no test compound wells by the 20 μM test compound well (FIG. 5B). The maleimide NanoBRET-590 tracer outperformed the other maleimide dye tracers tested, giving the largest compound knockdown at the lowest concentration. This differential reactivity was not anticipated because the maleimide warhead was kept constant while only the dye, and in some cases the linker, were varied. This suggests that the maleimide NanoBRET-590 tracer is unique in its enhanced reactivity.

SEQ ID NO: 1 -NANOLUC-
MKHHHHHHAIAMVFTLEDFVGDWRQTAGYNLDQVLEQGGVSSLFQNLGVSV
TPIQRIVLSGENGLKIDIHVIIPYEGLSGDQMGQIEKIFKVVYPVDDHHFK
VILHYGTLVIDGVTPNMIDYFGRPYEGIAVFDGKKITVTGTLWNGNKIIDE
RLINPDGSLLFRVTINGVTGWRLCERILAV
SEQ ID NO: 2 -LGBIT-
MVFTLEDFVGDWEQTAAYNLDQVLEQGGVSSLLQNLAVSVTPIQRIVRSGE
NALKIDIHVIIPYEGLSADQMAQIEEVFKVVYPVDDHHFKVILPYGTLVID
GVTPNMLNYFGRPYEGIAVFDGKKITVTGTLWNGNKIIDERLITPDGSMLF
RVTINSHHHHHH
SEQ ID NO: 3 -SMBIT-VTGYRLFEEIL
SEQ ID NO: 4 -LGTRIP-
MVFTLEDFVGDWEQTAAYNLDQVLEQGGVSSLLQNLAVSVTPIQRIVRSGE
NALKIDIHVIIPYEGLSADQMAQIEEVFKVVYPVDDHHFKVILPYGTLVID
GVTPNMLNYFGRPYEGIAVFDGKKITVTGTLWNGNKIIDERLITPD
SEQ ID NO: 5 -SMTRIP9-GSMLFRVTINS
SEQ ID NO: 6 -SMTRIP10-VSVSGWRLFKKIS
SEQ ID NO: 7 -HIBIT-VSGWRLFKKIS

1. A system comprising:
(a) a target protein tethered to a first component of a luminescent reporter; and
(b) a cysteine-reactive tracer comprising second component of the luminescent reporter tethered to a cysteine-reactive group.

2. The system of claim 1, wherein the first component of the luminescent reporter is a donor lumiphore and the second component of the luminescent reporter is an acceptor fluorophore, wherein light emitted at a first wavelength from the donor lumiphore is capable of exciting the acceptor fluorophore, resulting in emission of light from the acceptor fluorophore at a second wavelength.

3. The system of claim 2, wherein the donor lumiphore is a bioluminescent luciferase comprising at least 70% sequence identity with SEQ ID NO: 1.

4-5. (canceled)

6. The system of claim 2, further comprising a luminescent substrate for the donor lumiphore, wherein the luminescent substrate is an imidazopyrazine selected from coelenterazine, furimazine, and fluorofurimazine.

7-8. (canceled)

9. The system of claim 2, wherein the donor lumiphore is fluorescent.

10-11. (canceled)

12. The system of claim 2, wherein the acceptor fluorophore is a small molecule fluorophore and the cysteine-reactive group is selected from a maleimide moiety, an acrylamide moiety, a chloroacetamide moiety, a geminal dicyanocyclopropane carboxamide moiety, a bromo-dihydroisoxazole carboxamide moiety, and an azetidinyl oxadiazole moiety.

13. (canceled)

14. The system of claim 1, wherein the first component of the luminescent reporter is a first component of a bioluminescent complex and the second component of the luminescent reporter is a second component of the bioluminescent complex, wherein upon formation of the bioluminescent complex by binding of the first component to the second component light is emitted from the bioluminescent complex in the presence of a luminescent substrate.

15. The system of claim 14, wherein the first component of the bioluminescent complex comprises at least 70% sequence identity with SEQ ID NO: 2 and the second component of a bioluminescent complex comprises at least 70% sequence identity with SEQ ID NO: 3.

16-18. (canceled)

19. The system of claim 1, wherein the target protein and the cysteine-reactive tracer are within a cell or a cell lysate.

20. (canceled)

21. The system of claim 1, wherein the cysteine-reactive group of the cysteine-reactive tracer is selected from: a maleimide moiety, an acrylamide moiety, a chloroacetamide moiety, a geminal dicyanocyclopropane carboxamide moiety, a bromo-dihydroisoxazole carboxamide moiety, and an azetidinyl oxadiazole moiety, and wherein the second component of the luminescent reporter is a small molecule fluorophore selected from a rhodol, rhodamine, or BODIPY dye.

22-24. (canceled)

25. The system of claim 1, further comprising a cysteine-reactive test agent.

26. A method of quantifying the covalent binding of a cysteine-reactive test agent to a target protein, the method comprising:
(a) contacting a target protein tethered to a first component of a luminescent reporter with the cysteine-reactive test agent;
(b) contacting the target protein with a cysteine-reactive tracer comprising second component of the luminescent reporter tethered to a cysteine-reactive group,
(c) detecting a luminescent signal from the luminescent reporter, wherein the amount of the luminescent signal emitted is inversely proportional to the amount of the cysteine-reactive test agent covalently bound to the target protein.

27. The method of claim 26, wherein the target protein, the cysteine-reactive tracer, and the cysteine-reactive test agent are within a cell, wherein the target protein is present as a target fusion with the first component of the luminescent reporter expressed within the cell, and wherein contacting the target protein with the cysteine-reactive test agent comprises adding the cysteine-reactive test agent extracellularly and allowing the cysteine-reactive test agent to enter the cell.

28-30. (canceled)

31. The method of claim 27, wherein contacting the target protein with the cysteine-reactive tracer comprises (i) adding the cysteine-reactive tracer extracellularly and (ii) allowing the cysteine-reactive tracer to enter the cell.

32. The method of claim 26, wherein contacting the target protein with the cysteine-reactive tracer comprises (i) lysing the cell to produce a cell lysate and (ii) adding the cysteine-reactive tracer to the cell lysate.

33. The method of claim 26, wherein the first component of the luminescent reporter is a bioluminescent donor lumiphore that is a luciferase comprising at least 70% sequence identity with SEQ ID NO: 1, and the second component of the luminescent reporter is an acceptor fluorophore, wherein light emitted at a first wavelength from the donor lumiphore is capable of exciting the acceptor fluorophore, resulting in emission of light from the acceptor fluorophore at a second wavelength.

34-36. (canceled)

37. The method of claim 33, wherein detecting the luminescent signal comprises contacting the donor lumiphore with a luminescent substrate that is an imidazopyrazine selected from coelenterazine, furimazine, and fluorofurimazine.

38-47. (canceled)

48. The method of claim 26, wherein the cysteine-reactive group is selected from a maleimide moiety, an acrylamide moiety, a chloroacetamide moiety, a geminal dicyanocyclopropane carboxamide moiety, a bromo-dihydroisoxazole carboxamide moiety, and an azetidinyl oxadiazole moiety.

49. The method of claim 26, wherein the first component of the luminescent reporter is a first component of a bioluminescent complex and the second component of the luminescent reporter is a second component of the bioluminescent complex, wherein upon formation of the bioluminescent complex by binding of the first component to the second component light is emitted from the bioluminescent complex in the presence of a luminescent substrate, and wherein the first component of the bioluminescent complex comprises at least 70% sequence identity with SEQ ID NO: 2 and the second component of a bioluminescent complex comprises at least 70% sequence identity with SEQ ID NO: 3.

50-51. (canceled)

52. A method of quantifying the covalent binding of a cysteine-reactive test agent to a target protein within a cell, the method comprising:
(a) providing a cell comprising a target protein tethered to a first component of a luminescent reporter;
(b) contacting the cell with the cysteine-reactive test agent, and allowing the cysteine-reactive test agent to enter the cell and interact with the target protein;
(c) lysing the cell to produce a cell lysate;
(d) contacting the cell lysate with a cysteine-reactive tracer comprising second component of the luminescent reporter tethered to a cysteine-reactive group, and allowing the cysteine-reactive tracer to interact with the target protein;
(e) detecting a luminescent signal from the luminescent reporter, wherein the amount of the luminescent signal emitted is inversely proportional to the amount of the cysteine-reactive test agent covalently bound to the target protein.

53-55. (canceled)


SEQUENCE LISTING (7 sequences)

SEQ ID NO: 1 - protein, 183 aa, Synthetic construct
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 2 - protein, 165 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 3 - protein, 11 aa, Synthetic construct
VTGYRLFEEIL

SEQ ID NO: 4 - protein, 148 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 5 - protein, 11 aa, Synthetic construct
GSMLFRVTINS

SEQ ID NO: 6 - protein, 13 aa, Synthetic construct
VSVSGWRLFKKIS

SEQ ID NO: 7 - protein, 11 aa, Synthetic construct
VSGWRLFKKIS